(19) United States
(12) Patent Application Publication
(10) Pub. No.: US 2023/0348535 A1
(43) Pub. Date: Nov. 2, 2023

(54) RADIOMETAL-BINDING COMPOUNDS FOR DIAGNOSIS OR TREATMENT OF PROSTATE SPECIFIC MEMBRANE ANTIGEN-EXPRESSING CANCER

(71) Applicants: PROVINCIAL HEALTH SERVICES AUTHORITY, Vancouver (CA); THE UNIVERSITY OF BRITISH COLUMBIA, Vancouver (CA)

(72) Inventors: Kuo-Shyan LIN, Richmond (CA); François BÉNARD, Vancouver (CA); Hsiou-Ting KUO, Sherbrooke (CA); Zhengxing ZHANG, Vancouver (CA)

(21) Appl. No.: 18/219,458

(22) Filed: Jul. 7, 2023

Related U.S. Application Data
(63) Continuation of application No. 17/990,897, filed on Nov. 21, 2022, now abandoned.
Application No. 17/990,897 is a continuation of application No. 16/758,361, filed on Apr. 22, 2020, now abandoned.
(60) Provisional application No. 62/575,460, filed on Oct. 22, 2017.

(51) Int. Cl. C07K 5/02; A61K 51/04

(52) U.S. Cl. CPC C07K 5/0215; A61K 51/0455; A61K 51/048; A61K 51/0482; A61K 51/0485; C07K 5/021

(57) ABSTRACT

This application relates to compounds of Formula (I-a) or Formula (I-b), or is salts or solvates thereof. R¹ is —(CH₂)₅CH₃ or comprises 2-4 fused benzene rings. R² is I, Br, F, Cl, H, OH, OCH₃, NH₂, NO₂ or CH₃. R³ is a peptide-bonded glycine, aspartate or glutamate or is glutamate peptide bonded through C_{delta}. L is —CH₂NH—, —(CH₂)₂NH—, —(CH₂)₃NH—, or —(CH₂)₄NH—. R⁴ is a radiometal chelator optionally bound by a radiometal. Variable ‘n’ is 1-3. The compounds may be useful for imaging prostate specific membrane antigen (PSMA)-expressing tissues or for treating PSMA-expressing diseases (e.g. cancer).

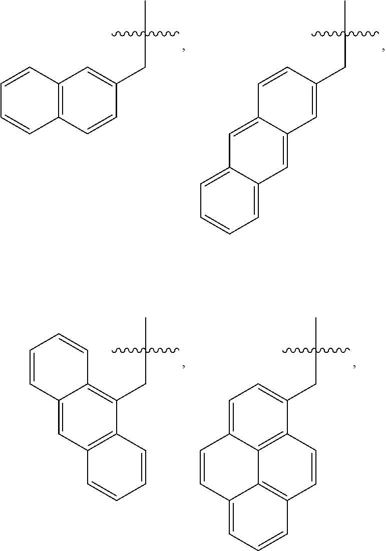 [SMILES: *Cc1ccc2ccccc2c1 ; *Cc1ccc2cc3ccccc3cc2c1 ; *Cc1c2ccccc2cc2ccccc12 ; *Cc1ccc2ccc3cccc4ccc1c2c34]

### FIELD OF INVENTION

The present invention relates to radiolabelled compounds for selective imaging or treatment of cancer, particularly compounds that target prostate specific membrane antigen.

### BACKGROUND OF THE INVENTION

Prostate specific membrane antigen (PSMA) is a transmembrane protein that catalyzes the hydrolysis of N-acetyl-aspartylglutamate to glutamate and N-acetylaspartate.¹ PSMA is not expressed in most normal tissues, but is overexpressed (up to 1,000-fold) in prostate tumors and metastases.²⁻³ Due to its pathological expression pattern, various radiolabeled PSMA-targeting constructs have been designed and evaluated for endoradiotherapy of prostate cancer.⁴⁻⁷

The common radiolabeled PSMA-targeting endoradiotherapeutic agents are derivatives of lysine-urea-glutamate (Lys-urea-Glu) including ¹³¹I-MIP-1095, ¹⁷⁷Lu-PSMA-617 and ¹⁷⁷Lu-PSMA I&T.⁵⁻⁷ Among them, ¹⁷⁷Lu-PSMA-617 is the most studied agent, and is currently being evaluated in multicenter trials.⁷⁻¹⁴ Preliminary data demonstrated that ¹⁷⁷Lu-PSMA-617 was effective in treating metastatic prostate cancer with 32-60% of patients having >50% reduction in PSA levels, and without severe side effects.⁷⁻¹³ In a phase 2 Australian study, an objective response was observed in 82% of patients with measurable nodal or visceral disease.¹⁴ However, the complete response rate was low (<7%), and up to 33% of the patients still had progressive disease after ¹⁷⁷Lu-PSMA-617 treatment.^{7,9-13} Interestingly, a recent report showed impressive responses with ²²⁵Ac-PSMA-617 (replacing ¹⁷⁷Lu with an α-emitter ²²⁵Ac) in advanced metastatic prostate cancer patients, including one subject whose disease had progressed despite ¹⁷⁷Lu-PSMA-617 therapy.¹⁵

Despite the great potential of ²²⁵Ac-PSMA-617 for endoradiotherapy, the supply of ²²⁵Ac is globally limited. More effective ¹⁷⁷Lu-labeled PSMA-targeting agents will have a greater immediate impact for endoradiotherapy of prostate cancer than ²²⁵Ac-PSMA-617 as good manufacturing practice (GMP) compliant ¹⁷⁷Lu is commercially available in larger quantities from multiple suppliers. The greater efficacy of ²²⁵Ac-PSMA-617 may be due to the high linear energy transfer of α-particles, which causes double strand breaks that may be less susceptible to radiation resistance compared to the indirect damage produced by p-particles emitted by ¹⁷⁷Lu. One approach to increase the radiotherapeutic efficacy is to increase the radiation dose deposited in tumors per unit administered radioactivity of the ¹⁷⁷Lu-labeled agents. Improving the delivery of ¹⁷⁷Lu to tumors can also reduce the cost of therapeutic radiopharmaceuticals by decreasing radioisotope costs.

No admission is necessarily intended, nor should it be construed, that any of the preceding information constitutes prior art against the present invention.

### SUMMARY OF THE INVENTION

Disclosed herein are novel compounds targeting the PSMA.

This disclosure provides a compound which is of Formula I-a or Formula I-b, or is a salt or solvate of Formula I-a or Formula I-b:

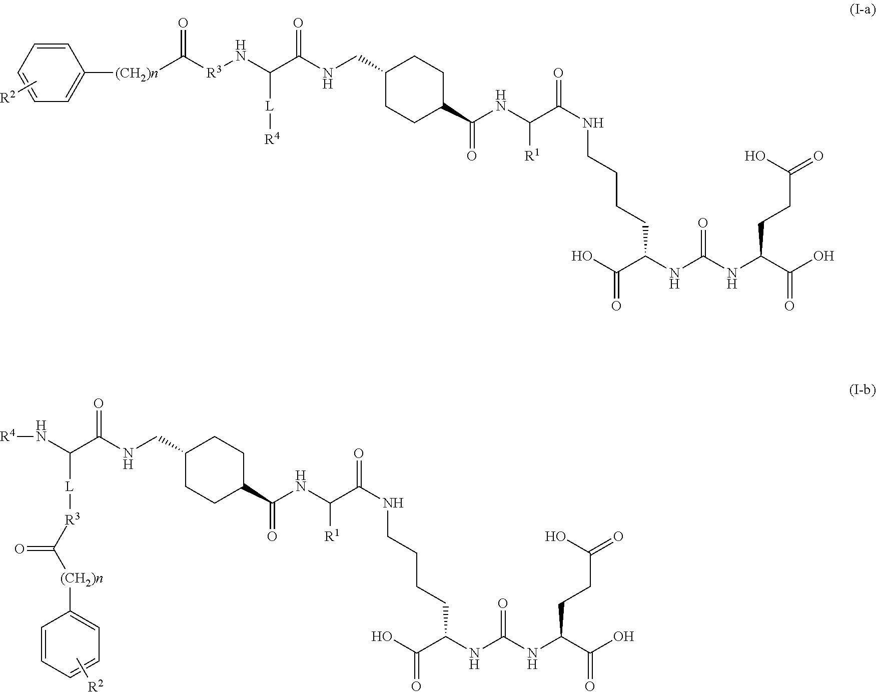

wherein:
R¹ is

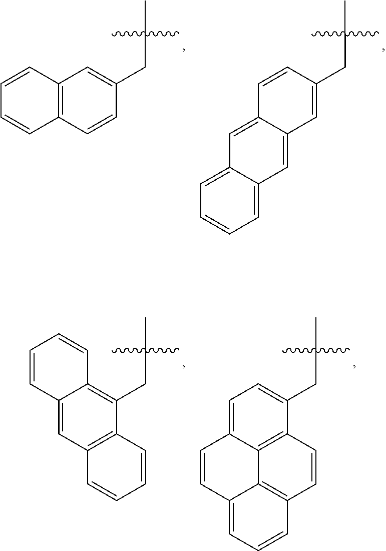 [SMILES: *Cc1ccc2ccccc2c1 ; *Cc1ccc2cc3ccccc3cc2c1 ; *Cc1c2ccccc2cc2ccccc12 ; *Cc1ccc2ccc3cccc4ccc1c2c34]

or —(CH₂)₅CH₃;
R² is I, Br, F, Cl, H, OH, OCH₃, NH₂, NO₂ or CH₃;
R³ is

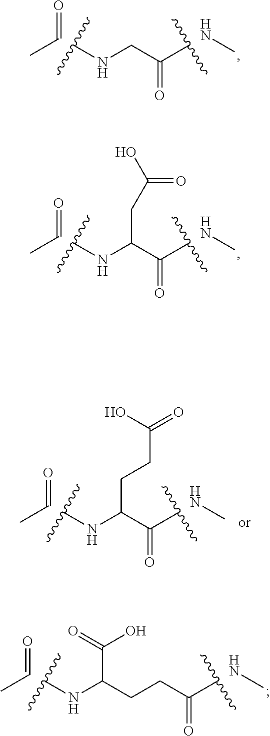

L is —CH₂NH—, —(CH₂)₂NH—, —(CH₂)₃NH—, or —(CH₂)₄NH—;
R⁴ is a radiometal chelator optionally bound by radiometal X; and
n is 1-3.

There is also disclosed a compound which has Formula II or is a salt or solvate of Formula II:

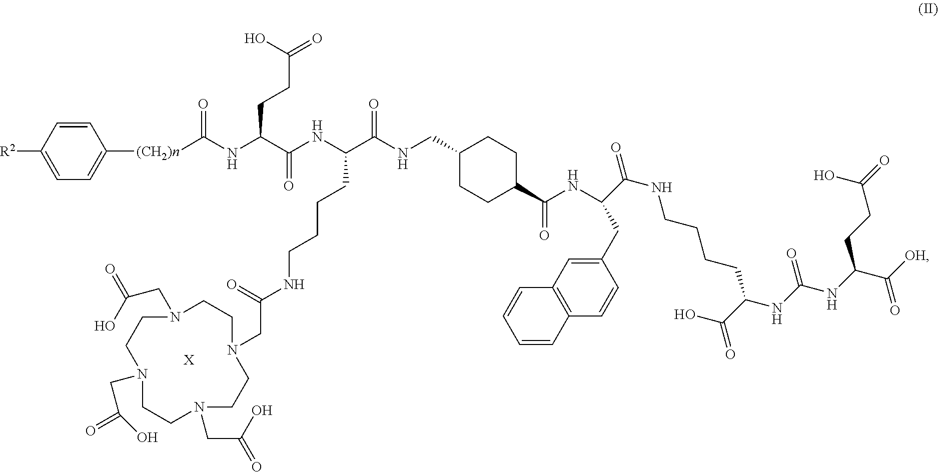

wherein: R² is I, Br or methyl; n is 1-3; and X is absent, ²²⁵Ac or ¹⁷⁷Lu.

In some embodiments, when X is a diagnostic radiometal (e.g. suitable for imaging, such as but not necessarily limited to ⁶⁴Cu, ¹¹¹In, ⁸⁹Zr, ⁴⁴Sc, ⁶⁸Ga, ^{99m}Tc, ⁸⁶Y, ¹⁵²Tb or ¹⁵⁵Tb), such compounds may be used for imaging PSMA-expressing cancer in a subject. Accordingly, there is also disclosed a method of imaging PSMA-expressing cancer in a subject, the method comprising: administering to the subject a composition comprising the compound and a pharmaceutically acceptable excipient; and imaging tissue of the subject.

In some embodiments, when X is a therapeutic radiometal (e.g. a toxic radiometal, such as but not limited to ⁶⁴Cu, ⁶⁷Cu, ⁹⁰Y, ¹¹¹In, ^{114m}In, ^{117m}Sn, ¹⁵³Sm, ¹⁴⁹Tb, ¹⁶¹Tb, ¹⁷⁷Lu, ²²⁵Ac, ²¹³Bi, ²²⁴Ra, ²¹²Bi, ²¹²Pb, ²²⁵Ac, ²²⁷Th, ²²³Ra, ⁴⁷Sc, ¹⁸⁶Re or ¹⁸⁸Re), such compounds may be used for treating PSMA-expressing cancer in a subject. Accordingly, there is also disclosed a method of treating prostate specific membrane antigen (PSMA)-expressing cancer in a subject, the method comprising: administering to the subject a composition comprising the compound and a pharmaceutically acceptable excipient.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows representative displacement curves of ¹⁸F-DCFPyL binding to LNCaP prostate cancer cells by Lu-PSMA-617 and Lu-HTK01169 from assays performed in triplicate.

FIG. 2 shows SPECT/CT images of (A)¹⁷⁷Lu-labeled PSMA-617 and (B) HTK01169 in mice bearing LNCaP tumor xenografts. Higher and sustained uptake of ¹⁷⁷Lu-HTK01169 in tumor xenografts was observed.

FIG. 3A is a graph showing biodistribution of ¹⁷⁷Lu-PSMA-617 for selected organs in mice bearing LNCaP tumor xenografts (n 5). Bars organized left to right: 1 h, 4 h, 24 h, 72 h and 120 h.

FIG. 3B is a graph showing biodistribution of ¹⁷⁷Lu-HTK01169 for selected organs in mice bearing LNCaP tumor xenografts (n 5). Bars organized left to right: 1 h, 4 h, 24 h, 72 h and 120 h.

FIG. 4 is a graph showing radiation doses (mGy/MBq) delivered by ¹⁷⁷Lu-HTK01169 (left bar) and ¹⁷⁷Lu-PSMA-617 (right bar) to major organs/tissues of a 25-g mouse calculated using the OLINDA software.

FIG. 5 is a graph showing radiation doses (mGy/MBq) of ¹⁷⁷Lu-PSMA-617 (lower) and ¹⁷⁷Lu-HTK01169 (upper) to LNCaP tumors calculated using the OLINDA software. These data were obtained with various tumor masses but assuming same tumor uptake (% ID, percent injected dose) and residence time for ¹⁷⁷Lu-PSMA-617 and ¹⁷⁷Lu-HTK01169.

FIG. 6 is a line graph showing overall survival for LNCaP tumor-bearing mice (n=8 per group) injected with saline (the control group), ¹⁷⁷Lu-PSMA-617 (18.5 MBq) or ¹⁷⁷Lu-HTK01169 (2.3-18.5 MBq). From shortest to longest median survival: control, 2.3 MBq ¹⁷⁷Lu-HTK01169, 18.5 MBq ¹⁷⁷Lu-PSMA-617, 4.6 MBq ¹⁷⁷Lu-HTK01169, 9.3 MBq ¹⁷⁷Lu-HTK01169, and 18.5 MBq ¹⁷⁷Lu-HTK01169.

FIG. 7 shows line graphs of changes of (A) tumor volume and (B) body weight over time after mice were treated with saline.

FIG. 8 shows line graphs of changes of (A) tumor volume and (B) body weight over time after mice were treated with ¹⁷⁷Lu-PSMA-617 (18.5 MBq).

FIG. 9 shows lines graphs of changes of (A) tumor volume and (B) body weight over time after mice were treated with ¹⁷⁷Lu-HTK01169 (18.5 MBq).

FIG. 10 shows lines graphs of changes of (A) tumor volume and (B) body weight overtime after mice were treated with ¹⁷⁷Lu-HTK01169 (9.3 MBq).

FIG. 11 shows lines graphs of changes of (A) tumor volume and (B) body weight overtime after mice were treated with ¹⁷⁷Lu-HTK01169 (4.6 MBq).

FIG. 12 shows lines graphs of changes of (A) tumor volume and (B) body weight overtime after mice were treated with ¹⁷⁷Lu-HTK01169 (2.3 MBq).

FIG. 13 shows maximum intensity projection PET/CT images of ⁶⁸Ga-HTK03026, ⁶⁸Ga-HTK03027, ⁶⁸Ga-HTK03029, and ⁶⁸Ga-HTK03041 acquired at 1 h or 3 h post-injection in mice bearing LNCaP tumor xenografts. All ⁶⁸Ga-labeled compounds are excreted mainly via the renal pathway. The tumor uptake of ⁶⁸Ga-HTK03026, ⁶⁸Ga-HTK03027 and ⁶⁸Ga-HTK03029 are comparable, whereas ⁶⁸Ga-HTK03041 has the highest tumor uptake which increases from 1 h to 3 h post-injection.

FIG. 14 shows maximum intensity projection PET/CT images of ⁶⁸Ga-HTK03055, ⁶⁸Ga-HTK03056, and ⁶⁸Ga-HTK03058 acquired at 1 h and 3 h post-injection in mice bearing LNCaP tumor xenografts. All three compounds show some extent of blood retention as heart is clearly visualized in images at 1 h post-injection. While uptake in blood (heart) decreases overtime (1 h to 3 h post-injection), uptake in tumors increases over time.

FIG. 15 shows maximum intensity projection PET/CT images of ⁶⁸Ga-HTK03082, ⁶⁸Ga-HTK03085, and ⁶⁸Ga-HTK03086 acquired at 1 h and 3 h post-injection in mice bearing LNCaP tumor xenografts. All three compounds are excreted mainly by the renal pathway. ⁶⁸Ga-HTK03085 and ⁶⁸Ga-HTK03086 shows significantly higher blood retention when compared to that of ⁶⁸Ga-HTK03082. The tumor uptake of ⁶⁸Ga-HTK03085 and ⁶⁸Ga-HTK03086 also increases over time from 1 h to 3 h post-injection.

FIG. 16 shows maximum intensity projection PET/CT images of ⁶⁸Ga-HTK03087, ⁶⁸Ga-HTK03089, and ⁶⁸Ga-HTK03090 acquired at 1 h and 3 h post-injection in mice bearing LNCaP tumor xenografts. ⁶⁸Ga-HTK03089 and ⁶⁸Ga-HTK03090 shows significantly higher blood retention when compared to that of ⁶⁸Ga-HTK03087. The tumor uptake of ⁶⁸Ga-HTK03089 and ⁶⁸Ga-HTK03090 also increases over time from 1 h to 3 h post-injection.

### DETAILED DESCRIPTION

As used herein, the terms “comprising,” “having”, “including” and “containing,” and grammatical variations thereof, are inclusive or open-ended and do not exclude additional, unrecited elements and/or method steps. The term “consisting essentially of” if used herein in connection with a composition, use or method, denotes that additional elements and/or method steps may be present, but that these additions do not materially affect the manner in which the recited composition, method or use functions. The term “consisting of” if used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the content clearly dictates otherwise. The use of the word “a” or “an” when used herein in conjunction with the term “comprising” may mean “one,” but it is also consistent with the meaning of “one or more,” “at least one” and “one or more than one.”

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, compound, etc.

As used herein, the terms “treat”, “treatment”, “therapeutic” and the like includes ameliorating symptoms, reducing disease progression, improving prognosis and reducing cancer recurrence.

As used herein, the term “diagnostic agent” includes an “imaging agent”. As such, a “diagnostic radiometal” includes radiometals that are suitable for use as imaging agents.

The term “subject” refers to an animal (e.g. a mammal or a non-mammal animal). The subject may be a human or a non-human primate. The subject may be a laboratory mammal (e.g., mouse, rat, rabbit, hamster and the like). The subject may be an agricultural animal (e.g., equine, ovine, bovine, porcine, camelid and the like) or a domestic animal (e.g., canine, feline and the like).

As used herein, the terms “salt” and “solvate” have their usual meaning in chemistry. As such, when the compound is a salt or solvate, it is associated with a suitable counter-ion. It is well known in the art how to prepare salts or to exchange counter-ions. Generally, such salts can be prepared by reacting free acid forms of these compounds with a stoichiometric amount of a suitable base (e.g. without limitation, Na, Ca, Mg, or K hydroxide, carbonate, bicarbonate, or the like), or by reacting free base forms of these compounds with a stoichiometric amount of a suitable acid. Such reactions are generally carried out in water or in an organic solvent, or in a mixture of the two. Counter-ions may be changed, for example, by ion-exchange techniques such as ion-exchange chromatography. All zwitterions, salts, solvates and counter-ions are intended, unless a particular form is specifically indicated.

In certain embodiments, the salt or counter-ion may be pharmaceutically acceptable, for administration to a subject. More generally, with respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington—The Science and Practice of Pharmacy, 21st edition (Gennaro et al editors. Lippincott Williams & Wilkins Philadelphia), each of which is incorporated by reference in its entirety.

In an aspect of the invention, there is disclosed a compound which is of Formula I-a or Formula I-b, or is a salt or solvate of Formula I-a or Formula I-b:

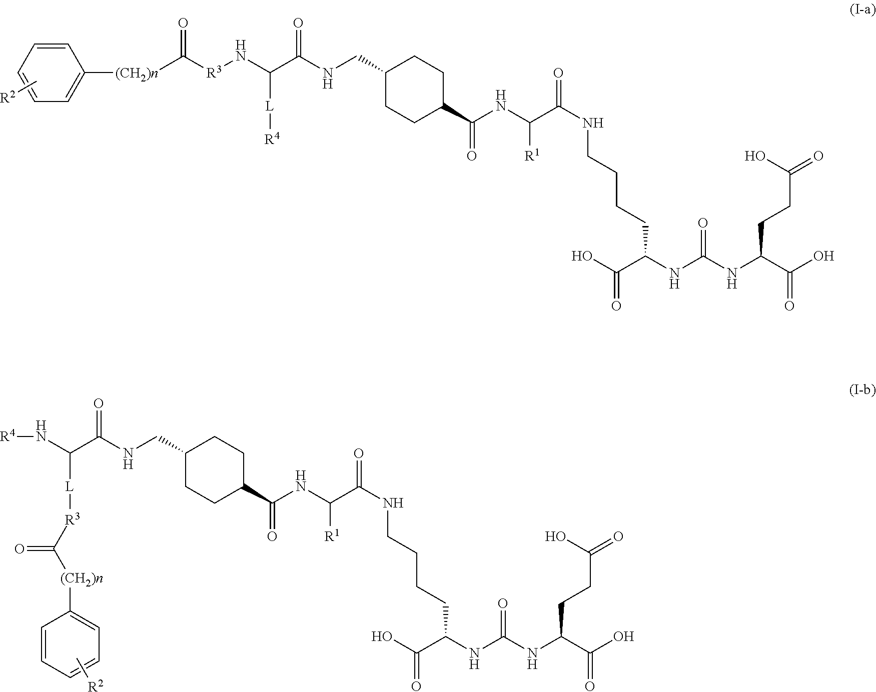

wherein:
R¹ is

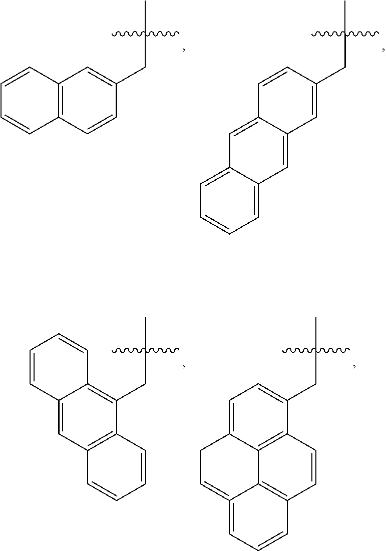

or —(CH₂)₅CH₃;
R² is I, Br, F, Cl, H, OH, OCH₃, NH₂, NO₂ or CH₃;
R³ is

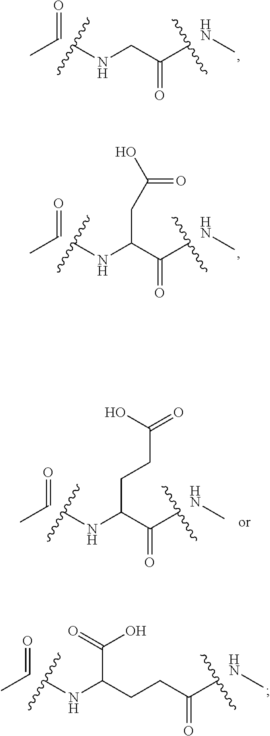

L is —CH₂NH—, —(CH₂)₂NH—, —(CH₂)₃NH—, or —(CH₂)₄NH—;
R⁴ is a radiometal chelator optionally bound by radiometal X; and
n is 1-3.

The wavy line “˜˜” symbol shown through a bond in a chemical formula (e.g. Formula I-a or Formula I-b) is intended to define the R group (e.g. R¹, R² and R³) on one side of the wavy line, without modifying the definition of the structure on the opposite side of the wavy line. Where an R group is bonded on two or more sides (e.g. R³), the atoms outside the wavy lines are include to clarify orientation of the R group. As such, only the atoms between the two wavy lines constitute the definition of the R group.

In some embodiments, the compound is of Formula I-a or is a salt or solvate of Formula I-a.

In some embodiments, the compound is of Formula I-b or is a salt or solvate of Formula I-b.

In some embodiments, R¹ is

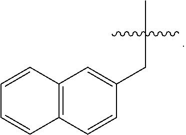 [SMILES: *Cc1ccc2ccccc2c1]

In some embodiments, R¹ is

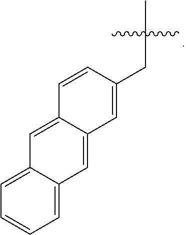 [SMILES: *Cc1ccc2cc3ccccc3cc2c1]

In some embodiments, R¹ is

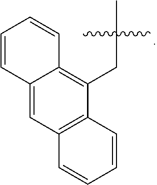 [SMILES: *Cc1c2ccccc2cc2ccccc12]

In some embodiments, R¹ is

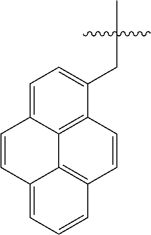 [SMILES: *Cc1ccc2ccc3cccc4ccc1c2c34]

In some embodiments, R¹ is —(CH₂)₅CH₃.

R¹ forms the side chain of an amino acid residue (e.g. 2-naphthylalanine etc.). In some embodiments, this amino acid is an L-amino acid, i.e.

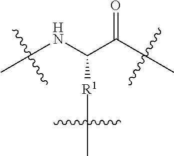

(e.g. L-2-naphthylalanine etc.). In some embodiment, the amino acid is a D-amino acid

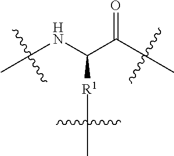

(e.g. D-2-naphthylalanine etc.).

In some embodiments, R¹ is

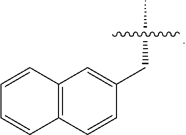

In some embodiments, R¹ is

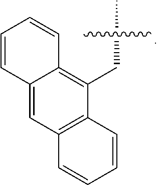

In some embodiments, R¹ is

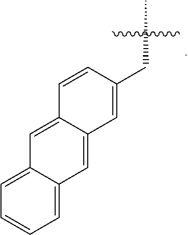

In some embodiments, R¹ is

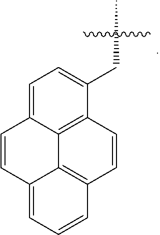

In some embodiments, n is 1. In some embodiments, n is 2. In some embodiments, n is 3.

As shown in Formulas I-a and I-b, there is a single R² group on the benzene ring. When not hydrogen, R² may be in para, meta or ortho position on the benzene ring, i.e.:

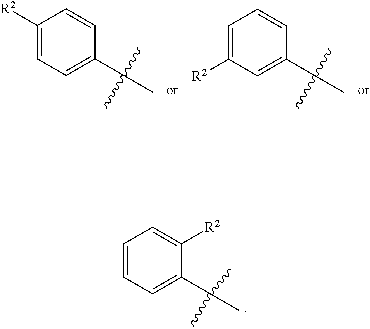

In some embodiments, R² is in para position. In some embodiments, R² is in meta position. In some embodiments, R² is in ortho position.

In some embodiments, R² is H. In some embodiments, R² is I. In some embodiments, R² is Br.

In some embodiments, R² is F. In some embodiments, R² is Cl. In some embodiments, R² is OH. In some embodiments, R² is OCH₃. In some embodiments, R² is NH₂. In some embodiments, R² is NO₂. In some embodiments, R² is CH₃.

In some embodiments, R³ is

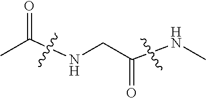

(i.e. a Gly residue).

In some embodiments, R³ is

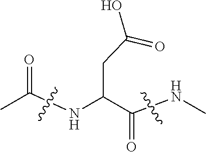

(i.e. an Asp residue). In some embodiments, the Asp residue is D-Asp. In some embodiments, the Asp is L-Asp.

In some embodiments, R³ is

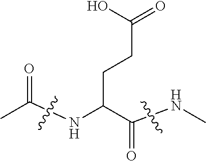

(i.e. a Glu residue). In some embodiments, the Glu residue is D-Glu. In some embodiments, the Glu residue is L-Glu.

In some embodiments, R³ is

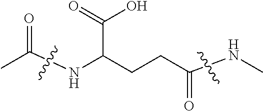

In some embodiments, R³ is

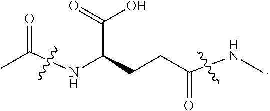

In some embodiments, R³ is

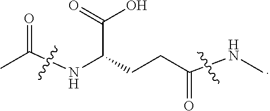

R⁴ may be any radiometal chelator suitable for binding to the radiometal of interest (i.e. X) and which is functionalized for attachment to an amino group. Many suitable radiometal chelators are known, e.g. as summarized in Price and Orvig, Chem. Soc. Rev., 2014, 43, 260-290, which is incorporated by reference in its entirety. In some embodiments, R⁴ is:
DOTA (1,4,7,10-tetraazacyclododecane-1,4,7,10-tetraacetic acid) or a derivative thereof, such as but not limited to DOTAGA;
TETA (1,4,8,11-tetraazacyclotetradecane-1,4,8,11-tetraacetic acid) or a derivative thereof, such as but not limited to CB-TE2A (4,11-bis-(carboxymethyl)-1,4,8,11-tetraazabicyclo[6.6.2]-hexadecane);
SarAr (1-N-(4-Aminobenzyl)-3,6,10,13,16,19-hexaazabicyclo[6.6.6]-eicosane-1,8-diamine or a derivative thereof;
NOTA (1,4,7-triazacyclononane-1,4,7-triacetic acid) or a derivative thereof, such as but not limited to NODAGA;
TRAP (1,4,7-triazacyclononane-1,4,7-tris[methyl(2-carboxyethyl)phosphinic acid) or a derivative thereof;
HBED (N,N0-bis(2-hydroxybenzyl)-ethylenediamine-N,N0-diacetic acid) or a derivative thereof;
2,3-HOPO (3-hydroxypyridin-2-one) or a derivative thereof;
PCTA (3,6,9,15-tetraazabicyclo[9.3.1]-pentadeca-1(15),11,13-triene-3,6,9-triacetic acid) or a derivative thereof;
DFO (desferrioxamine) or a derivative thereof, such as but not limited to tetrahydroxamate DFO* (DFO-star);
DTPA (diethylenetriaminepentaacetic acid) or a derivative thereof, such as but not limited to CHX-DTPA (2-(p-isothiocyanatobenzyl)-cyclohexyldiethylenetriaminepentaacetic acid);
OCTAPA (N,N0-bis(6-carboxy-2-pyridylmethyl)-ethylenediamine-N,N0-diacetic acid) or a derivative thereof (e.g. picolinic acid derivatives); or
H2-MACROPA (N,N′-bis[(6-carboxy-2-pyridil)methyl]-4,13-diaza-18-crown-6) or a derivative thereof.

In some embodiments, X is absent.

In some embodiments, X is a therapeutic radiometal. For example, but without limitation, X may be ⁶⁴Cu, ⁶⁷Cu, ⁹⁰Y, ¹¹¹In, ^{114m}In, ^{117m}Sn, ¹⁵³Sm, ¹⁴⁹Tb, ¹⁶¹Tb, ¹⁷⁷Lu, ²²⁵Ac, ²¹³Bi, ²²⁴Ra, ²¹²Bi, ²¹²Pb, ²²⁵Ac, ²²⁷Th, ²²³Ra, ⁴⁷Sc, ¹⁸⁶Re, or ¹⁸⁸Re. In some embodiments, X is ⁶⁴Cu. In some embodiments, X is ⁶⁷Cu. In some embodiments, X is ⁹⁰Y. In some embodiments, X is ¹¹¹In. In some embodiments, X is ^{114m}In. In some embodiments, X is ^{117m}Sn. In some embodiments, X is ¹⁵³Sm. In some embodiments, X is ¹⁴⁹Tb. In some embodiments, X is ¹⁶¹Tb. In some embodiments, X is ¹⁷⁷Lu. In some embodiments, X is ²²⁵Ac. In some embodiments, X is ²¹³Bi. In some embodiments, X is ²²⁴Ra. In some embodiments, X is ²¹²Bi. In some embodiments, X is ²¹²Pb. In some embodiments, X is ²²⁵Ac. In some embodiments, X is ²²⁷Th. In some embodiments, X is ²²³Ra. In some embodiments, X is ⁴⁷Sc. In some embodiments, X is ¹⁸⁶Re. In some embodiments, X is ¹⁸⁸Re.

In some embodiments, X is a diagnostic radiometal. For example, but without limitation, X may be ⁶⁴Cu, ¹¹¹In, ⁸⁹Zr, ⁴⁴Sc, ⁶⁸Ga, ^{99m}Tc, ⁸⁶Y, ¹⁵²Tb or ¹⁵⁵Tb. In some embodiments, X is ⁶⁴Cu. In some embodiments, X is ¹¹¹In. In some embodiments, X is ⁸⁹Zr. In some embodiments, X is ⁴⁴Sc. In some embodiments, X is ⁶⁸Ga. In some embodiments, X is ^{99m}Tc. In some embodiments, X is ⁸⁶Y. In some embodiments, X is ¹⁵²Tb. In some embodiments, X is ¹⁵⁵Tb.

In some embodiments, R¹ is

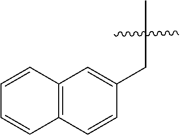 [SMILES: *Cc1ccc2ccccc2c1]

### and R³ is

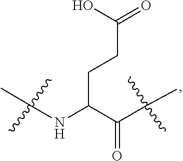

wherein R² is I, Br, F, Cl, H, OH, OCH₃, NH₂, NO₂ or CH₃, and wherein X is absent, ⁹⁰Y, ⁶⁷Ga, ⁶⁸Ga, ¹⁷⁷Lu, ²²⁵Ac, or ¹¹¹In. In certain of these embodiments, R² is in para position. In certain of these embodiments, R² is I. In certain of these embodiments, X is ¹⁷⁷Lu, and in other embodiments, X is ²²⁵Ac.

In some embodiments, R¹ is

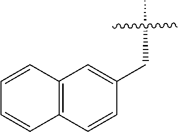

### and R³ is

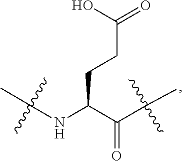

wherein R² is I, Br, F, Cl, H, OH, OCH₃, NH₂, NO₂ or CH₃, and wherein X is absent, ⁹⁰Y, ⁶⁷Ga, ⁶⁸Ga, ¹⁷⁷Lu, ²²⁵Ac, or ¹¹¹In. In certain of these embodiments, R² is in para position. In certain of these embodiments, R² is I. In certain of these embodiments, X is ¹⁷⁷Lu, and in other embodiments, X is ²²⁵Ac. In certain of these embodiments, n is 3.

In some embodiments, L is —CH₂NH—. In some embodiments, L is —(CH₂)₂NH—. In some embodiments, L is —(CH₂)₃NH—. In some embodiments, L is —(CH₂)₄NH—.

L forms the side chain of an amino acid residue (e.g. 2,3-diaminopropionic acid (Dap), 2,4-diaminobutanoic acid (Dab), ornithine (Orn) or lysine (Lys)). In some embodiments, this amino acid is an L-amino acid, i.e.

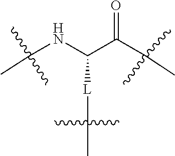

(e.g. L-Dap, L-Dab, L-Orn or L-Lys). In some embodiment, the amino acid is a D-amino acid

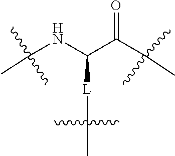

(e.g. D-Dap, D-Dab, D-Orn or D-Lys).

In some embodiments, the amino acid residue formed by L is an L-amino acid and the amino acid residue formed by R¹ is also an L-amino acid. In some embodiments, the amino acid residue formed by L is a D-amino acid and the amino acid residue formed by R¹ is also a D-amino acid. In some embodiments, the amino acid residue formed by L is an L-amino acid and the amino acid residue formed by R¹ is a D-amino acid. In some embodiments, the amino acid residue formed by L is a D-amino acid and the amino acid residue formed by R¹ is an L-amino acid.

In some embodiments, the compound has Formula II or is a salt or solvate of Formula II:

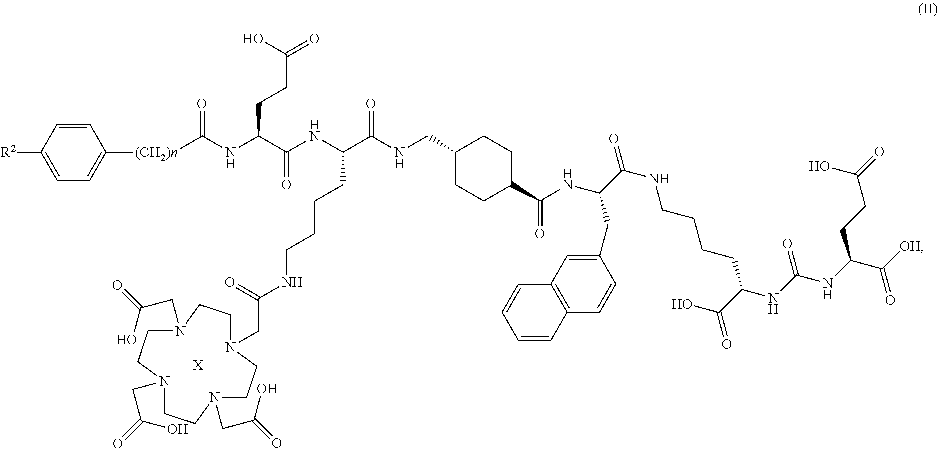

wherein: R² is I, Br or methyl; n is 1-3; and X is absent, ²²⁵Ac or ¹⁷⁷Lu. In some of these embodiments, R² is I. In some of these embodiments, R² is Br. In some of these embodiments, R² is methyl. In some of these embodiments, n is 1. In some of these embodiments, n is 2. In some of these embodiments, n is 3. In some of these embodiments, X is absent. In some of these embodiments, X is ¹⁷⁷Lu and is bound in the DOTA group. In some of these embodiments, X is ²²⁵Ac and is bound in the DOTA group.

In some embodiments, the compound has Formula III or is a salt or solvate of Formula III:

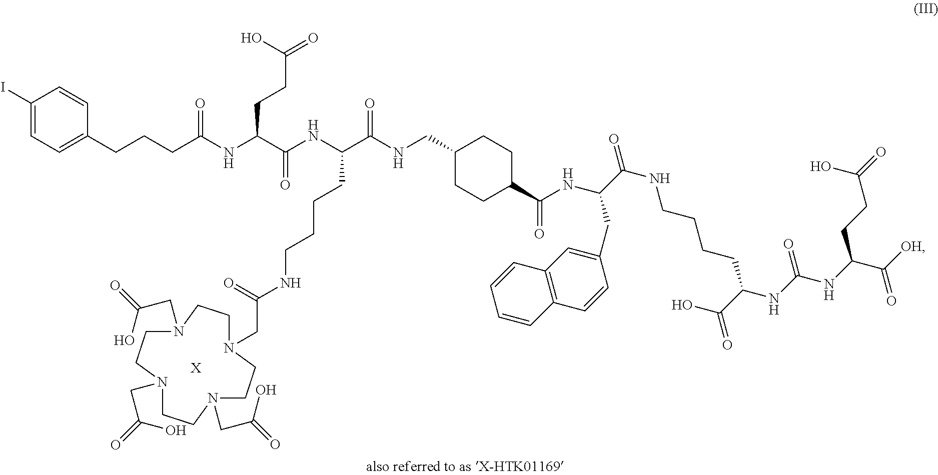

wherein X is absent, ⁹⁰Y, ⁶⁷Ga, ⁶⁸Ga, ¹⁷⁷Lu, ²²⁵Ac, or ¹¹¹In. When X in is ¹⁷⁷Lu, the compound has the structure shown below, or is a salt or solvate thereof:

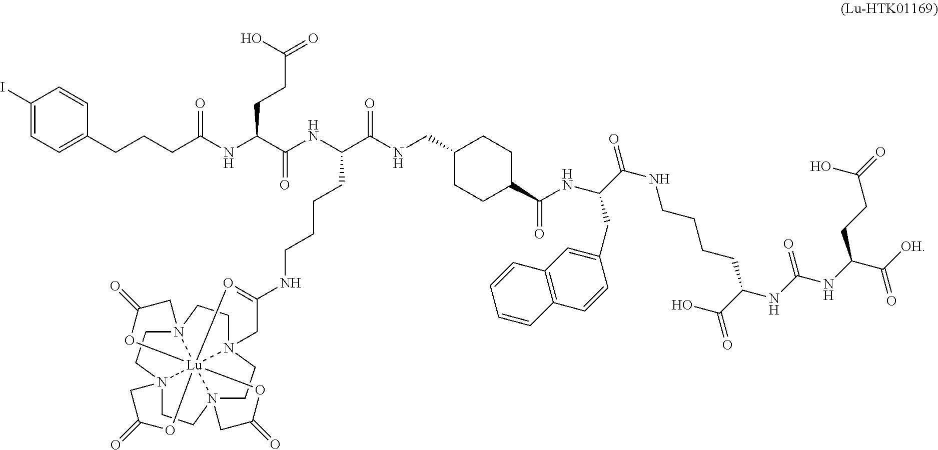

A synthetic scheme for HTK01169 and Lu-HTK01169 is provided in Example 1 below. Example 2 provides synthetic schemes for preparing a number of metal-chelating PSMA-binding compounds which incorporate many of the options for the R groups of Formulas I-a and I-b.

The above compounds modulate albumin-binding and PSMA-binding (e.g. as compared to Lu-PSMA-617) to modulate (e.g. enhance) tumor uptake/retention, so as to provide alternative or improved diagnostic or therapeutic agents for PSMA-expressing cancers. In particular, the above compounds comprise an albumin-binding domain, i.e.

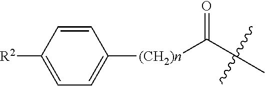

(e.g. iodophenylbutyryl group in Lu-HTK01169; see also PCT Patent Publication No. WO 2008/053360), which increases blood circulation time of the compound. Modifying the albumin-binding group by varying the R² and/or the value of n (i.e. n=1, 2 or 3) and/or introducing R³ (e.g. addition of Gly or a carboxylate-containing Asp or Glu) can modulate (increase or decrease) the albumin-binding strength (i.e. binding affinity) of the compound and thereby modulate the resulting blood circulation time of the compound. Without wishing to be bound by theory, a compound with too strong binding to albumin (i.e. too high binding affinity to albumin) would retain the compound too long in blood circulation and accumulation in the tumor will be very low. This would result in lower overall uptake in the tumor and too high a radiation dose to bone marrow. At the same time, if the albumin binding affinity is too weak, the compound will clear from blood circulation too fast, reducing the chance to accumulate in the tumor. In addition, the above compounds also comprise a Lys-ureido-Glu PSMA-binding moeity. The PSMA-binding strength of the compound can be modulated (increased or decreased) by modifying R¹. Without wishing to be bound by theory, the modulated tumor uptake/retension of the above compounds may be due to the modulated albumin-binding and/or PSMA-binding strengths (e.g. as compared to Lu-PSMA-617). The diagnostic or therapeutic efficacy may be further modulated by varying the chelator and bound radiometal. As demonstrated by the following Examples, the above variables have been tuned in the above compounds to enhance PSMA-expressing tumor uptake/retention and therefore diagnostic or therapeutic efficacy.

When X is a diagnostic radiometal, there is disclosed use of certain embodiments of the compound for preparation of a radiolabelled tracer for imaging PSMA-expressing tissues in a subject. There is also disclosed a method of imaging PSMA-expressing tissues in a subject, in which the method comprises: administering to the subject a composition comprising certain embodiments of the compound and a pharmaceutically acceptable excipient; and imaging tissue of the subject, e.g. using positron emission tomography (PET). When the tissue is a diseased tissue (e.g. a PSMA-expressing cancer), PSMA-targeted treatment may then be selected for treating the subject.

When X is a therapeutic radiometal, there is disclosed use of certain embodiments of the compound (or a pharmaceutical composition thereof) for the treatment of PSMA-expressing diseases (e.g. cancer) in a subject. Accordingly, there is provided use of the compound in preparation of a medicament for treating PSMA-expressing disease in a subject. There is also provided a method of treating PSMA-expressing disease in a subject, in which the method comprises: administering to the subject a composition comprising the compound and a pharmaceutically acceptable excipient. For example, but without limitation, the disease may be a PSMA-expressing cancer.

PSMA expression has been detected in various cancers (e.g. Rowe et al., 2015, Annals of Nuclear Medicine 29:877-882; Sathekge et al., 2015, Eur J Nucl Med Mol Imaging 42:1482-1483; Verburg et al., 2015, Eur J Nucl Med Mol Imaging 42:1622-1623; and Pyka et al., J Nucl Med Nov. 19, 2015jnumed.115.164442). Accordingly, without limitation, the PSMA-expressing cancer may be prostate cancer, renal cancer, breast cancer, thyroid cancer, gastric cancer, colorectal cancer, bladder cancer, pancreatic cancer, lung cancer, liver cancer, brain tumor, melanoma, neuroendocrine tumor, ovarian cancer or sarcoma. In some embodiments, the cancer is prostate cancer.

The present invention will be further illustrated in the following examples.

### Example 1: ¹⁷⁷Lu-HTK01169

1.1 Materials and Methods

1.11 General Methods

All chemicals and solvents were obtained from commercial sources, and used without further purification. Human serum for protein binding assay was obtained from Innovative Research (Novi, MI). PSMA-617 and HTK01169 were synthesized using a solid phase approach on an Aapptec (Louisville, KY) Endeavor 90 peptide synthesizer. Mass analyses were performed using an AB SCIEX (Framingham, MA) 4000 QTRAP mass spectrometer system with an ESI ion source. Purification and quality control of non-radioactive and ¹⁷⁷Lu-labeled peptides were performed on Agilent (Santa Clara, CA) HPLC systems equipped with a model 1200 quaternary pump and a model 1200 UV absorbance detector. The radio-HPLC system was equipped with a Bioscan (Washington, DC) NaI scintillation detector. The HPLC columns used were a Phenomenex (Torrance, CA) semi-preparative column (Luna C18, 5μ, 250×10 mm) and a Phenomenex analytical column (Luna C18, 5μ, 250×4.6 mm). Radioactivity of ¹⁷⁷Lu-labeled peptides was measured using a Capintec (Ramsey, NJ) CRC®-25R/W dose calibrator.

1.12 Solid-Phased Synthesis of PSMA-617 and HTK01169

Synthesis of PSMA-617 and its albumin-binder-containing derivative HTK01169 was modified from reported procedures,¹⁶ starting from Fmoc-Lys(ivDde)-Wang resin. After coupling the isocyanate of the t-butyl-protected glutamyl moiety,¹⁷ the ivDde-protecting group was removed with 2% hydrazine in N,N-dimethylformamide (DMF). Subsequent coupling of Fmoc-2-NaI—OH, Fmoc-tranexamic acid and DOTA-tris(t-bu)ester, followed by trifluoroacetic acid (TFA) cleavage provided the crude product of PSMA-617. After HPLC purification using the semi-preparative column with 25% acetonitrile in water containing 0.1% TFA at a flow rate of 4.5 mL/min (t_{R}=10.5 min), PSMA-617 was obtained in 25% yield. ESI-MS: calculated [M+H]⁺ for PSMA-617 C₄₉H₇₂N₉O₁₆ 1042.5; found [M+H]⁺ 1042.6.

For the synthesis of HTK01169, Fmoc-Lys(ivDde)-OH was coupled to the sequence after Fmoc-tranexamic acid. Elongation was continued with the addition of Fmoc-Glu(tBu)-OH and 4-(p-iodophenyl)butyric acid to the N-terminus. Subsequently, the ivDde-protecting group was removed with 2% hydrazine in DMF, and DOTA-tris(t-bu)ester was coupled to the Lys side chain. The peptide was cleaved with TFA treatment, and purified by HPLC using the semi-preparative column with 37% acetonitrile in water containing 0.1% TFA at a flow rate of 4.5 mL/min (t_{R}=9.7 min). The yield of HTK01169 was 21%. ESI-MS: calculated [M+H]⁺ for HTK01169 C₇₀H₁₀N₁₂O₂₁I, 1571.6; found [M+H]⁺ 1571.7.

1.13 Synthesis of Lu-PSMA-617 and Lu-HTK01169

A solution of PSMA-617 (5.5 mg, 5.3 μmol) or HTK01169 (4.1 mg, 2.6 μmol) was incubated with LuCl₃ (5 equivalents) in NaOAc buffer (0.1 M, 500 μL, pH 4.2) at 90° C. for 15 min, and then purified by HPLC using the semi-preparative column. For Lu-PSMA-617, the HPLC conditions were 25% acetonitrile in water with 0.1% TFA at a flow rate of 4.5 mL/min (t_{R}=9.7 min). The yield was 62%. ESI-MS: calculated [M+H]⁺ for Lu-PSMA-617 C₄₉H₆₉N₉O₁₆[Lu] 1214.4; found [M+H]⁺ 1214.4. For Lu-HTK01169, the HPLC conditions were 37% acetonitrile in water with 0.1% TFA at a flow rate of 4.5 mL/min (t_{R}=10.0 min). The yield was 31%. ESI-MS: calculated [M+H]⁺ for Lu-HTK01169 C₇₀H₉₇N₁₂O₂₁I[Lu] 1743.5; found [M+H]⁺ 1743.9.

1.14 In vitro Competition Binding Assay

In vitro competition binding assays were conducted as previously reported using LNCaP prostate cancer cells and ¹⁸F-DCFPyL as the radioligand.¹⁸ Briefly, LNCaP cells (400,000/well) were plated onto a 24-well poly-D-lysine coated plate for 48 h. Growth media was removed and replaced with HEPES buffered saline (50 mM HEPES, pH 7.5, 0.9% sodium chloride) and the cells were incubated for 1 h at 37° C. ¹⁸F-DCFPyL (0.1 nM) was added to each well (in triplicate) containing various concentrations (0.5 mM-0.05 nM) of tested compounds (Lu-PSMA-617 or Lu-HTK01169). Non-specific binding was determined in the presence of 10 μM non-radiolabeled DCFPyL. The assay mixtures were further incubated for 1 h at 37° C. with gentle agitation. Then, the buffer and hot ligand were removed, and cells were washed twice with cold HEPES buffered saline. To harvest the cells, 400 μL of 0.25% trypsin solution was added to each well. Radioactivity was measured on a PerkinElmer (Waltham, MA) Wizard2 2480 automatic gamma counter. Nonlinear regression analyses and Ki calculations were performed using the GraphPad Prism 7 software.

1.15 Synthesis of ¹⁷⁷Lu-PSMA-617 and ¹⁷⁷Lu-HTK01169

¹⁷⁷LuCl₃ (329.3-769.9 MBq in 10-20 μL) was added to a solution of PSMA-617 or HTK01169 (25 μg) in NaOAc buffer (0.5 mL, 0.1 M, pH 4.5). The mixture was incubated at 90° C. for 15 min, and then purified by HPLC. The HPLC purification conditions (semi-prep column, 4.5 mL/min) for ¹⁷⁷Lu-PSMA-617 and ¹⁷⁷Lu-HTK01169 were 23% and 36% acetonitrile in water (0.1% TFA), respectively. The retention times for ¹⁷⁷Lu-PSMA-617 and ¹⁷⁷Lu-HTK01169 were 15.0 min and 13.8 min, respectively. Quality control was performed on the analytical column with a flow rate of 2 mL/min using the corresponding purification solvent conditions. The retention times for ¹⁷⁷Lu-PSMA-617 and ¹⁷⁷Lu-HTK01169 were both around 5.5 min.

1.16 Plasma Protein Binding Assay

Plasma protein binding assays were performed according to literature methods.¹⁹ Briefly, 37 kBq of ¹⁷⁷Lu-PSMA-617 or ¹⁷⁷Lu-HTK01169 in 50 μL PBS was added into 200 μL human serum and the mixture was incubated at room temperature for 1 min. The mixture was then loaded onto a membrane filter (Nanosep®, 30 K, Pall Corporation, USA) and centrifuged for 45 min (30,130×g). Saline (50 μL) was added and centrifugation was continued for another 15 min. The top part with the membrane filter and the bottom part with the solution were counted on a gamma counter. For control, saline was used in place of human serum.

1.17 SPECT/CT Imaging, Biodistribution and Endoradiotherapy Studies

SPECT/CT imaging and biodistribution were performed using NOD-scid IL2Rgamma^{null} (NSG) male mice, and the endoradiotherapy study was conducted using NOD.Cg-Rag1^{tm1Mom} Il2rg^{tm1Wj1}/SzJ (NRG) male mice. The mice were maintained and the experiments were conducted in according to the guidelines established by the Canadian Council on Animal Care and approved by Animal Ethics Committee of the University of British Columbia. Mice were anesthetized by inhalation with 2% isoflurane in oxygen, and implanted subcutaneously with 1×10⁷ LNCaP cells posterior to the left shoulder. Mice were used for studies when the tumor reached 5-8 mm in diameter 5-6 weeks after inoculation.

SPECT/CT imaging experiments were conducted using the MILabs (Utrecht, the Netherlands) U-SPECT-II/CT scanner. Each tumor-bearing mouse was injected with ˜37 MBq of ¹⁷⁷Lu-labeled PSMA-617 or HTK01169 through the tail vein under anesthesia (2% isoflurane in oxygen). The mice were allowed to recover and roam freely in their cage and imaged at 4, 24, 72 and 120 hours after injection. At each time point, the mice were sedated again and positioned in the scanner. A 5-min CT scan was conducted first for anatomical reference with a voltage setting at 60 kV and current at 615 ρA followed by a 60-min static emission scan acquired in list mode using an ultra-high resolution multi-pinhole rat-mouse (1 mm pinhole size) collimator. Data were reconstructed using the U-SPECT II software with a 20% window width on three energy windows. The photopeak window was centered at 208 keV, with lower scatter and upper scatterwindows centered at 170 and 255 keV, respectively. The images were reconstructed using the ordered subset expectation maximization algorithm (3 iterations, 16 subsets), and a 0.5 mm post-processing Gaussian filter. Images were decay corrected to injection time in PMOD (PMOD Technologies, Switzerland) then converted to DICOM for qualitative visualization in the Inveon Research Workplace software (Siemens Medical Solutions USA, Inc.).

For biodistribution studies, the mice were injected with ¹⁷⁷Lu-labeled PSMA-617 or HTK01169 (2-4 MBq) as described above. At predetermined time points (1, 4, 24, 72, or 120 h post-injection), the mice were euthanized by CO₂ inhalation. Blood was withdrawn immediately from the heart, and the organs/tissues of interest were collected. The collected organs/tissues were weighed and counted using an automated gamma counter. For the blocking study, mice were co-injected with ¹⁷⁷Lu-HTK01169 (2-4 MBq) and 50 nmol of the non-radioactive standard, and organs/tissues of interest were collected at 4 h post-injection.

For radiotherapy study, tumor-bearing mice were injected with saline (the control group), ¹⁷⁷Lu-PSMA-617 (18.5 MBq) or ¹⁷⁷Lu-HTK01169 (18.5, 9.3, 4.6, or 2.3 MBq) (n=8 per group). Tumor size and body weight were measured twice a week from the date of injection (Day 0) until completion of the study (Day 120). Endpoint criteria were defined as >20% weight loss, tumor volume >1000 mm³, or active ulceration of the tumor.

1.18 Radiation Dosimetry Calculation

Internal dosimetry estimates were calculated using the organ level internal dose assessment (OLINDA) software v.2.0.³⁷ These estimates were performed for the mouse using the 25 g MOBY phantom,³⁸ for humans using the NURBS model for the adult male,³⁹ and for the tumors using the previously reported unit density sphere model.⁴⁰ All the phantoms and the sphere model are available in OLINDA and require the input of the total number of decays normalized by injected activity in units of MBq×h/MBq for each of the source organ/tumor.

The biodistribution data (available in the Tables 1 and 2, below) was used to determine the kinetics input values required by OLINDA. First, each of the values was decayed to its corresponding time point (the values on the table are shown at injection time). Then the different time-points of the uptake data (% ID/g) for each organ were fitted to both mono-exponential

$$( \frac{\% ID}{g} = {ae}^{- bt} )$$

and bi-exponential

$$( \frac{\% ID}{g} = {ae}^{- bt} + {ce}^{- dt} )$$

functions using in-house software developed in Python. The best fit was selected based on maximizing the coefficient of determination (R2) of the fit and minimizing the residuals. The areas under the curves were analytically calculated based on the parameters obtained from the best fit of each organ and this provided the kinetic input values required by OLINDA.

In the mouse case, the adrenals, blood, fat, muscle, and seminal vesicles are not modeled in the phantom. These organs were grouped together and included in what OLINDA calls the remainder of the body.

Extrapolation of the mice biodistribution data to humans was performed using the method proposed by Kirschner et al.⁴¹ and shown in the following equation:

$${( \frac{\% ID}{{m}_{organ}} )}_{human} = {( \frac{\% ID}{{m}_{organ}} )}_{mouse} [ \frac{{M}_{mouse}}{{M}_{human}} \times {( {m}_{organ} )}_{human} ]$$

Where m_{organ} is the mass of the organ and M represents the total body mass. The subscripts indicate whether the values correspond to human or mouse. Masses for the organs and total body weight were taken from the simulated masses of the phantoms in OLINDA. As the biodistribution data does not differentiate between left colon, right colon, and rectum that are present in the OLINDA human phantom, it was assumed that these three regions of the intestine have the same activity uptake (% ID/g) as the large intestine of the biodistribution. The % ID/g of the blood was assumed to be the one for the heart contents of the phantom. This value was also used to calculate the bone marrow uptake based on the method described by Wessels et al.⁴² in which we assumed a hematocrit fraction of 0.40 based on the patient values shown on that study. At the end, red marrow values used the blood measurements scaled by a factor of 0.32. In the human case, the fat, muscle, and seminal vesicles that are present in the biodistribution data are not modelled in the phantom so the numbers of decays present in these regions were included in the remainder of the body. The data was again fitted as for the mouse case and the values for the total number of decays in units of MBq×h/MBq were inputted in OLINDA.

Lastly, the numbers of decays in the tumors were also calculated based on the biodistribution data of the mice and the values were inputted into the sphere model available in OLINDA.

1.2 Results

1.21 Peptide Synthesis and Radiochemistry

PSMA-617 and HTK01169 were synthesized in 25 and 21% yields, respectively. After reacting with LuCl₃ followed by HPLC purification, Lu-PSMA-617 and Lu-HTK01169 were obtained in 62 and 31% yields, respectively. The identities of PSMA-617, HTK01169 and their Lu complexes were confirmed by MS analyses.

¹⁷⁷Lu labeling was conducted in acetate buffer (pH 4.5) at 90° C. followed by HPLC purification. ¹⁷⁷Lu-PSMA-617 was obtained in 86.0±1.7% (n=3) radiochemical yield with 782±43.3 GBq/μmol molar activity and >99% radiochemical purity. ¹⁷⁷Lu-HTK01169 was obtained in 63.0±16.2% (n=4) radiochemical yield with 170±73.6 GBq/μmol molar activity and >99% radiochemical purity.

1.22 Binding to PSMA and Serum Proteins

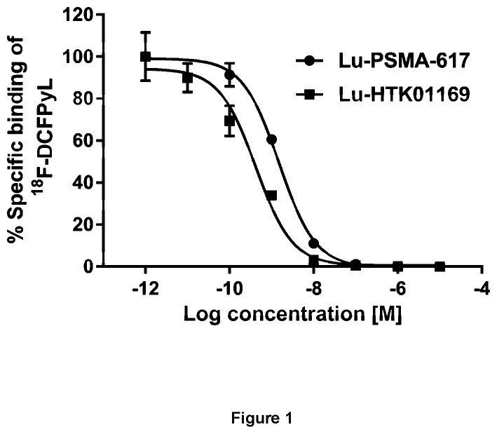

Lu-PSMA-617 and Lu-HTK01169 inhibited the binding of ¹⁸F-DCFPyL to PSMA on LNCaP cells in a dose dependent manner (FIG. 1), and their calculated Ki values were 0.24±0.06 and 0.04±0.01 nM (n=3), respectively. After incubating with saline and centrifugation, the filter-bound radioactivities were 5.21±1.42 and 25.8±3.42% (n=3) for ¹⁷⁷Lu-PSMA-617 and ¹⁷⁷Lu-HTK01169, respectively. Replacing saline with human serum increased the filter-bound radioactivities to 82.7±0.32 and 99.2±0.02% (n=3) for ¹⁷⁷Lu-PSMA-617 and ¹⁷⁷Lu-HTK01169, respectively, under the same conditions.

1.23 SPECT/CT Imaging and Biodistribution

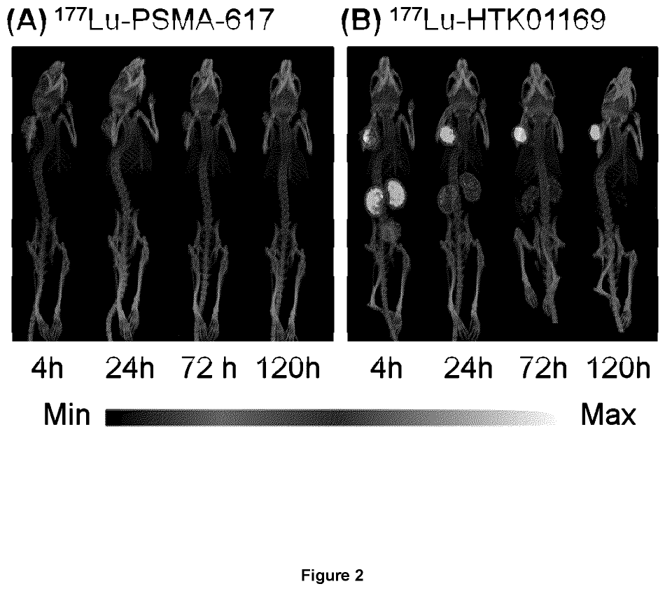

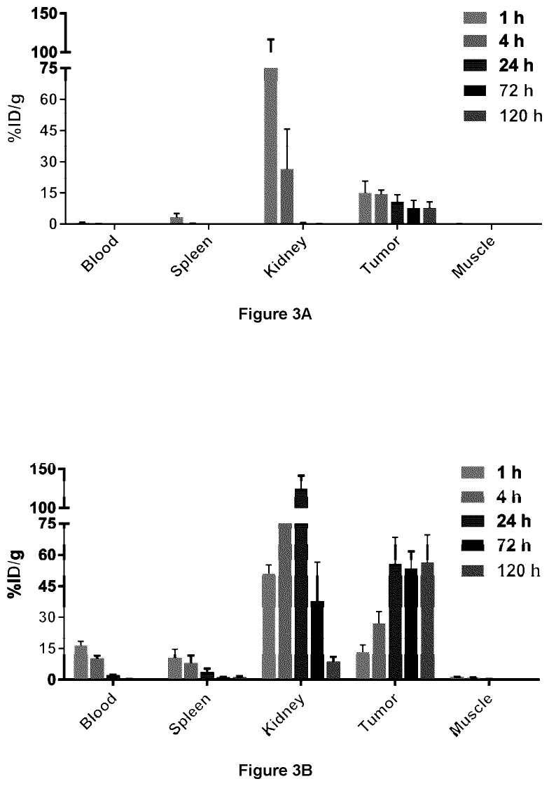

SPECT/CT imaging studies showed that both ¹⁷⁷Lu-PSMA-617 and ¹⁷⁷Lu-HTK01169 were excreted mainly via the renal pathway with higher renal retention of ¹⁷⁷Lu-HTK01169 especially at early time points (4 and 24 h, FIG. 2). Higher and sustained tumor uptake was observed for ¹⁷⁷Lu-HTK01169. The biodistribution data of ¹⁷⁷Lu-PSMA-617 and ¹⁷⁷Lu-HTK01169 are shown in FIGS. 3A and 3B (also Tables 1 and 2). These data were consistent with the observations from SPECT/CT images.

TABLE 1
Biodistribution data of ¹⁷⁷Lu-PSMA-617 in mice bearing LNCaP xenografts.

| Tissue | 1 h | 4 h | 24 h | 72 h | 120 h |
|---|---|---|---|---|---|
| (% ID/g) | (n = 5) | (n = 5) | (n = 5) | (n = 5) | (n = 6) |
| Blood | 0.68 ± 0.23 | 0.12 ± 0.18 | 0.00 ± 0.00 | 0.00 ± 0.00 | 0.00 ± 0.00 |
| Fat | 0.46 ± 0.18 | 0.16 ± 0.11 | 0.08 ± 0.03 | 0.05 ± 0.02 | 0.03 ± 0.01 |
| Seminal | 0.14 ± 0.08 | 0.02 ± 0.01 | 0.01 ± 0.00 | 0.01 ± 0.01 | 0.00 ± 0.00 |
| Testes | 0.49 ± 0.22 | 0.06 ± 0.01 | 0.02 ± 0.01 | 0.01 ± 0.00 | 0.01 ± 0.00 |
| Intestine | 0.26 ± 0.16 | 0.07 ± 0.04 | 0.02 ± 0.00 | 0.01 ± 0.01 | 0.03 ± 0.03 |
| Stomach | 0.18 ± 0.14 | 0.04 ± 0.02 | 0.04 ± 0.02 | 0.03 ± 0.03 | 0.05 ± 0.05 |
| Spleen | 3.34 ± 1.77 | 0.24 ± 0.14 | 0.05 ± 0.01 | 0.03 ± 0.02 | 0.02 ± 0.01 |
| Liver | 0.26 ± 0.15 | 0.08 ± 0.06 | 0.03 ± 0.01 | 0.02 ± 0.01 | 0.01 ± 0.00 |
| Pancreas | 0.33 ± 0.18 | 0.05 ± 0.01 | 0.01 ± 0.00 | 0.01 ± 0.01 | 0.00 ± 0.00 |
| Adrenal glands | 4.88 ± 2.41 | 0.40 ± 0.18 | 0.06 ± 0.03 | 0.10 ± 0.06 | 0.04 ± 0.02 |
| Kidneys | 97.2 ± 19.4 | 26.6 ± 19.1 | 0.58 ± 0.22 | 0.22 ± 0.11 | 0.08 ± 0.03 |
| Lung | 1.34 ± 0.39 | 0.16 ± 0.07 | 0.03 ± 0.01 | 0.02 ± 0.01 | 0.01 ± 0.00 |
| Heart | 0.28 ± 0.10 | 0.04 ± 0.02 | 0.01 ± 0.00 | 0.01 ± 0.01 | 0.00 ± 0.00 |
| Tumor | 15.1 ± 5.58 | 14.5 ± 1.83 | 10.9 ± 3.30 | 7.80 ± 3.69 | 7.91 ± 2.82 |
| Muscle | 0.18 ± 0.06 | 0.05 ± 0.03 | 0.01 ± 0.00 | 0.01 ± 0.00 | 0.00 ± 0.00 |
| Bone | 0.14 ± 0.06 | 0.06 ± 0.03 | 0.03 ± 0.02 | 0.05 ± 0.02 | 0.02 ± 0.01 |
| Brain | 0.03 ± 0.01 | 0.02 ± 0.00 | 0.01 ± 0.00 | 0.01 ± 0.00 | 0.01 ± 0.00 |
| Tumor/muscle | 86.9 ± 26.6 | 371 ± 171 | 1582 ± 353 | 1378 ± 335 | 4388 ± 4211 |
| Tumor/blood | 22.8 ± 6.85 | 290 ± 175 | 2293 ± 651 | 2206 ± 337 | 4425 ± 1485 |
| Tumor/kidney | 0.15 ± 0.04 | 0.79 ± 0.48 | 20.7 ± 8.39 | 38.0 ± 13.4 | 103 ± 75.9 |

TABLE 2
Biodistribution data of ¹⁷⁷Lu-HTK01169 in mice bearing LNCaP xenografts.

| | | | 4 h | | | |
|---|---|---|---|---|---|---|
| Tissue | 1 h | 4 h | Blocking | 24 h | 72 h | 120 h |
| (% ID/g) | (n = 6) | (n = 6) | (n = 5) | (n = 6) | (n = 6) | (n = 6) |
| Blood | 16.6 ± 1.85 | 10.2 ± 1.40 | 0.96 ± 0.25 | 2.10 ± 0.41 | 0.28 ± 0.10 | 0.06 ± 0.03 |
| Fat | 3.20 ± 1.03 | 2.95 ± 0.81 | 0.25 ± 0.13 | 2.06 ± 0.71 | 1.34 ± 0.72 | 0.73 ± 0.13 |
| Seminal | 1.02 ± 0.15 | 1.07 ± 0.62 | 0.19 ± 0.13 | 0.36 ± 0.13 | 0.12 ± 0.04 | 0.08 ± 0.04 |
| Testes | 2.14 ± 0.31 | 1.91 ± 0.63 | 0.58 ± 0.14 | 1.19 ± 0.23 | 0.62 ± 0.11 | 0.40 ± 0.05 |
| Intestine | 1.64 ± 0.20 | 1.25 ± 0.17 | 0.25 ± 0.10 | 0.45 ± 0.13 | 0.13 ± 0.06 | 0.10 ± 0.06 |
| Stomach | 0.50 ± 0.15 | 0.57 ± 0.11 | 0.19 ± 0.10 | 0.34 ± 0.12 | 0.17 ± 0.12 | 0.21 ± 0.20 |
| Spleen | 10.6 ± 4.07 | 8.05 ± 3.62 | 0.61 ± 0.17 | 3.70 ± 1.67 | 1.13 ± 0.32 | 1.18 ± 0.55 |
| Liver | 2.55 ± 0.45 | 1.97 ± 0.05 | 0.51 ± 0.08 | 1.03 ± 0.22 | 0.46 ± 0.12 | 0.34 ± 0.23 |
| Pancreas | 1.77 ± 0.24 | 1.51 ± 0.28 | 0.19 ± 0.02 | 0.59 ± 0.12 | 0.15 ± 0.02 | 0.07 ± 0.02 |
| Adrenal glands | 9.94 ± 2.10 | 9.91 ± 2.55 | 0.68 ± 0.24 | 4.53 ± 1.10 | 1.65 ± 0.46 | 0.65 ± 0.21 |
| Kidneys | 51.1 ± 4.07 | 85.7 ± 7.11 | 5.50 ± 1.95 | 125 ± 16.4 | 37.8 ± 18.7 | 8.63 ± 2.51 |
| Lung | 7.11 ± 0.98 | 5.92 ± 0.22 | 0.89 ± 0.29 | 1.98 ± 0.44 | 0.51 ± 0.17 | 0.17 ± 0.04 |
| Heart | 3.93 ± 0.95 | 2.50 ± 0.54 | 0.41 ± 0.07 | 0.83 ± 0.25 | 0.28 ± 0.04 | 0.16 ± 0.07 |
| Tumor | 13.4 ± 3.26 | 27.2 ± 5.56 | 1.70 ± 0.28 | 55.9 ± 12.5 | 53.6 ± 8.06 | 56.4 ± 13.2 |
| Muscle | 1.39 ± 0.12 | 0.98 ± 0.06 | 0.12 ± 0.02 | 0.39 ± 0.07 | 0.11 ± 0.02 | 0.04 ± 0.01 |
| Bone | 0.93 ± 0.31 | 0.79 ± 0.30 | 0.12 ± 0.04 | 0.26 ± 0.05 | 0.11 ± 0.03 | 0.08 ± 0.01 |
| Brain | 0.18 ± 0.07 | 0.16 ± 0.01 | 0.03 ± 0.00 | 0.06 ± 0.01 | 0.03 ± 0.00 | 0.03 ± 0.00 |
| Tumor/muscle | 9.76 ± 2.65 | 28.0 ± 6.92 | 13.7 ± 1.47 | 142 ± 14.4 | 519 ± 91.5 | 1708 ± 718 |
| Tumor/blood | 0.81 ± 0.18 | 2.71 ± 0.68 | 1.81 ± 0.31 | 26.7 ± 2.79 | 204 ± 63.5 | 1146 ± 722 |
| Tumor/kidney | 0.26 ± 0.05 | 0.32 ± 0.07 | 0.32 ± 0.06 | 0.45 ± 0.13 | 1.63 ± 0.58 | 7.12 ± 3.11 |

¹⁷⁷Lu-PSMA-617 cleared rapidly from blood and nontarget organs/tissues. At 1 h post-injection, there was only 0.68±0.23% ID/g left in blood. Uptake was observed in PSMA-expressing tissues including spleen (3.34±1.77% ID/g), adrenal glands (4.88±2.41% ID/g), kidneys (97.2±19.4% ID/g), lung (1.34±0.39% ID/g) and LNCaP tumors (15.1±5.58% ID/g).²⁰⁻²¹ The tumor uptake decreased gradually to 7.91±2.82% ID/g at 120 h post-injection. Due to faster clearance from other tissues/organs, the tumor-to-background contrast ratios of ¹⁷⁷Lu-PSMA-617 improved overtime (Table 1, above).

With a built-in albumin binder, the blood clearance of ¹⁷⁷Lu-HTK01169 was relatively slower than ¹⁷⁷Lu-PSMA-617 (FIGS. 3A and 3B). The tumor uptake of ¹⁷⁷Lu-HTK01169 increased continuously at early time points, peaked at 24 h post-injection (55.9±12.5% ID/g), and was sustained over the course of the study (56.4±13.2% ID/g at 120 h). Similar to ¹⁷⁷Lu-PSMA-617, uptake was also observed in the spleen, adrenal glands, kidneys, and lung (Table 2, above). The tumor-to-background contrast ratios of ¹⁷⁷Lu-PSMA-617 improved over time as well, due to sustained uptake in tumor and relatively faster clearance from other organs/tissues. Compared with the biodistribution data collected at the same time point (4 h), blocking with the cold standard reduced uptake in all collected tissues/organs especially the PSMA-expressing kidneys (125±16.4 vs 5.50±1.95% ID/g) and LNCaP tumors (55.9±12.5 vs 1.70±0.28% ID/g).

1.24 Radiation Dosimetry Calculations

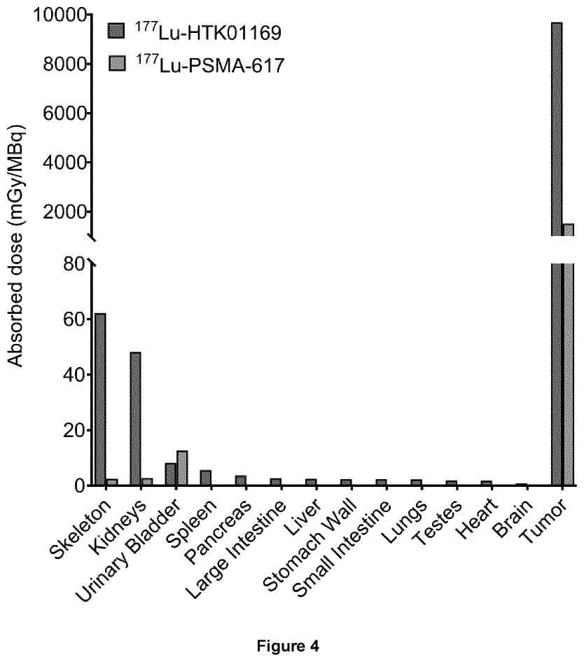

Based on the biodistribution data obtained from tumor-bearing mice, an estimate of radiation doses delivered to major organs/tissues of mice was calculated using the OLINDA software. The results are shown in FIG. 4 and Table 3 where both the input kinetics of the source organs calculated from the data fit (MBq-h/MBq), and the doses to the target organs (mGy/MBq) are presented. Compared to ¹⁷⁷Lu-PSMA-617, ¹⁷⁷Lu-HTK01169 delivered 9.4- to 23.1-fold higher radiation doses to all major organs except urinary bladder which received 1.5-fold higher radiation dose from ¹⁷⁷Lu-PSMA-617.

TABLE 3
Radiation doses (mGy/GBq) calculated for the major organs of 25-g mice using the OLINDA software.

| Kinetics value [MBq-h/MBq] | | | Organ doses [mGy/MBq] | | |
|---|---|---|---|---|---|
| | ¹⁷⁷Lu- | ¹⁷⁷Lu- | | ¹⁷⁷Lu- | ¹⁷⁷Lu- |
| Source organ | PSMA-617 | HTK01169 | Target organ | PSMA-617 | HTK01169 |
| Brain | 6.96E−03 | 3.21E−02 | Brain | 5.19E−02 | 9.06E−01 |
| Large intestine | 1.74E−02 | 2.25E−01 | Large | 2.10E−01 | 2.66E+00 |
| contents | | | intestine | | |
| Small intestine | 5.20E−02 | 6.73E−01 | Small intestine | 1.69E−01 | 2.35E+00 |
| Stomach contents | 5.42E−03 | 1.54E−02 | Stomach wall | 1.81E−01 | 2.41E+00 |
| Heart contents | 4.21E−03 | 1.66E−01 | Heart | 7.72E−02 | 1.79E+00 |
| Kidneys | 1.13E+00 | 1.94E+01 | Kidneys | 2.82E+00 | 4.82E+01 |
| Liver | 6.54E−02 | 1.41E+00 | Liver | 1.31E−01 | 2.50E+00 |
| Lungs | 5.10E−03 | 1.28E−01 | Lungs | 1.07E−01 | 2.32E+00 |
| Pancreas | 5.44E−03 | 1.30E−01 | Pancreas | 2.08E−01 | 3.64E+00 |
| Cortical bone | 9.96E−02 | 5.48E−01 | Skeleton | 2.48E+00 | 6.22E+01 |
| Spleen | 1.50E−02 | 4.38E−01 | Spleen | 2.58E−01 | 5.62E+00 |
| Testes | 4.96E−03 | 1.66E−01 | Testes | 2.07E−01 | 1.95E+00 |
| Thyroid | — | — | Thyroid | 5.94E−02 | 1.25E+00 |
| Urinary bladder | 1.10E+00 | 6.02E−01 | Urinary bladder | 1.26E+01 | 8.25E+00 |
| contents | | | wall | | |
| Remainder of | 6.82E−01 | 1.73E+01 | Remainder of | 1.77E−01 | 2.21E+00 |
| the body | | | the body | | |

Similar results were obtained for calculated radiation doses delivered to human organs/tissues (Table 4). Most human organs/tissues would receive 11.9- to 24.9-fold higher radiation doses from ¹⁷⁷Lu-HTK01169. Notably, the brain, heart, red marrow, and spleen would receive 6.0-, 50.4-, 30.4- and 28.1-fold higher doses with ⁷⁷Lu-HTK01169. The urinary bladder would receive 1.3-fold higher radiation dose from ¹⁷⁷Lu-PSMA-617.

TABLE 4
Radiation doses (mGy/GBq) calculated for the major organs of humans
(male) using the OLINDA software.

| Kinetics value [MBq-h/MBq] | | | Organ doses [mGy/MBq] | | |
|---|---|---|---|---|---|
| | ¹⁷⁷Lu- | ¹⁷⁷Lu- | | ¹⁷⁷Lu- | ¹⁷⁷Lu- |
| Source organ | PSMA-617 | HTK01169 | Target organ | PSMA-617 | HTK01169 |
| Adrenals | 1.00E−03 | 1.55E−02 | Adrenals | 7.64E−03 | 1.21E−01 |
| Brain | 6.71E−03 | 3.42E−02 | Brain | 4.32E−04 | 2.59E−03 |
| Esophagus | — | — | Esophagus | 7.60E−04 | 1.70E−02 |
| Eyes | — | — | Eyes | 6.80E−04 | 1.52E−02 |
| Gallbladder | — | — | Gallbladder | 8.76E−04 | 1.88E−02 |
| contents | | | wall | | |
| Left colon | 1.53E−03 | 1.99E−02 | Left colon | 1.81E−03 | 3.06E−02 |
| Small Intestine | 6.65E−03 | 8.61E−02 | Small intestine | 1.72E−03 | 2.85E−02 |
| Stomach contents | 5.06E−03 | 1.44E−02 | Stomach Wall | 1.69E−03 | 2.02E−02 |
| Right colon | 1.53E−03 | 1.99E−02 | Right colon | 1.31E−03 | 2.38E−02 |
| Rectum | 7.16E−04 | 9.27E−03 | Rectum | 1.45E−03 | 2.21E−02 |
| Heart contents | 4.29E−03 | 3.16E−01 | — | — | — |
| Heart wall | 2.02E−03 | 7.97E−02 | Heart wall | 1.00E−03 | 5.04E−02 |
| Kidneys | 3.98E−01 | 6.81E+00 | Kidneys | 1.11E−01 | 1.90E+00 |
| Liver | 2.32E−02 | 4.98E−01 | Liver | 1.37E−03 | 2.87E−02 |
| Lungs | 2.41E−02 | 6.03E−01 | Lungs | 1.81E−03 | 4.51E−02 |
| Pancreas | 8.55E−04 | 2.05E−02 | Pancreas | 7.60E−04 | 1.69E−02 |
| Prostate | — | — | Prostate | 1.17E−03 | 1.70E−02 |
| Salivary glands | — | — | Salivary glands | 6.98E−04 | 1.56E−02 |
| Red marrow | 3.15E−03 | 2.46E−01 | Red marrow | 7.59E−04 | 2.31E−02 |
| Cortical bone | 5.30E−03 | 1.03E−02 | Osteogenic Cells | 8.30E−04 | 1.54E−02 |
| Trabecular bone | — | — | — | — | — |
| Spleen | 6.93E−03 | 2.03E−01 | Spleen | 4.42E−03 | 1.24E−01 |
| Testes | 3.72E−04 | 1.25E−02 | Testes | 1.05E−03 | 3.17E−02 |
| Thymus | — | — | Thymus | 7.19E−04 | 1.64E−02 |
| Thyroid | — | — | Thyroid | 7.11E−04 | 1.60E−02 |
| Urinary bladder | 3.15E−01 | 1.72E−01 | Urinary bladder | 6.63E−02 | 5.19E−02 |
| contents | | | wall | | |
| Remainder of | 5.39E−01 | 1.21E+01 | Remainder of | 1.70E−03 | 2.70E−02 |
| the body | | | the body | | |

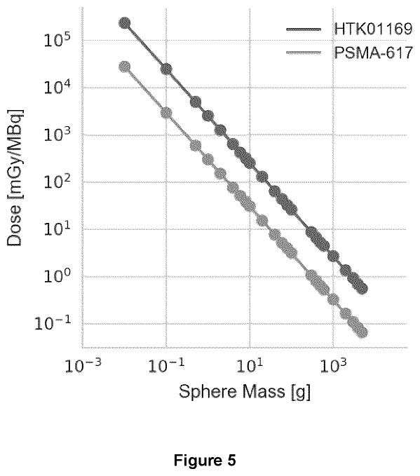

The behavior of radiation doses delivered to unit density spheres based on the kinetics of LNCaP tumors from ¹⁷⁷Lu-PSMA-617 and ¹⁷⁷Lu-HTK01169 are shown in FIG. 5 and Table 5. The kinetic uptake values used as input in OLINDA were 3.80 MBq-h/MBq and 31.72 MBq-h/MBq for ¹⁷⁷Lu-PSMA-617 and ¹⁷⁷Lu-HTK01169, respectively. ¹⁷⁷Lu-HTK01169 delivered an 8.3-fold higher radiation dose to LNCaP tumors than ¹⁷⁷Lu-PSMA-617 regardless of simulated sphere (tumor) sizes.

TABLE 5
Radiation dose (mGy/MBq) calculated from unit
density sphere models for the LNCaP tumors.

| Sphere/Tumor | | |
|---|---|---|
| volume (ml) | ¹⁷⁷Lu-PSMA-617 | ¹⁷⁷Lu-HTK01169 |
| 0.01 | 2.80E+04 | 2.33E+05 |
| 0.1 | 2.93E+03 | 2.44E+04 |
| 0.5 | 5.94E+02 | 4.96E+03 |
| 1 | 3.00E+02 | 2.50E+03 |
| 2 | 1.50E+02 | 1.25E+03 |
| 4 | 7.55E+01 | 6.30E+02 |
| 6 | 5.05E+01 | 4.21E+02 |
| 8 | 3.79E+01 | 3.16E+02 |
| 10 | 3.04E+01 | 2.53E+02 |
| 20 | 1.52E+01 | 1.27E+02 |
| 40 | 7.65E+00 | 6.38E+01 |
| 60 | 5.11E+00 | 4.26E+01 |
| 80 | 3.84E+00 | 3.20E+01 |
| 100 | 3.08E+00 | 2.57E+01 |
| 300 | 1.04E+00 | 8.64E+00 |
| 400 | 7.79E−01 | 6.50E+00 |
| 500 | 6.25E−01 | 5.21E+00 |
| 600 | 5.22E−01 | 4.35E+00 |
| 1000 | 3.15E−01 | 2.63E+00 |
| 2000 | 1.60E−01 | 1.33E+00 |
| 3000 | 1.07E−01 | 8.94E−01 |
| 4000 | 8.09E−02 | 6.74E−01 |
| 5000 | 6.50E−02 | 5.42E−01 |
| 6000 | 5.44E−02 | 4.53E−01 |

1.25 Endoradiotherapy Studies

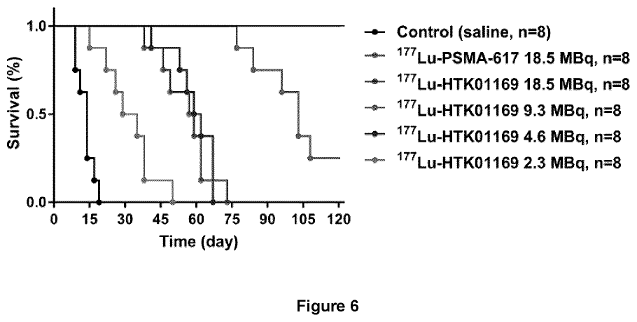

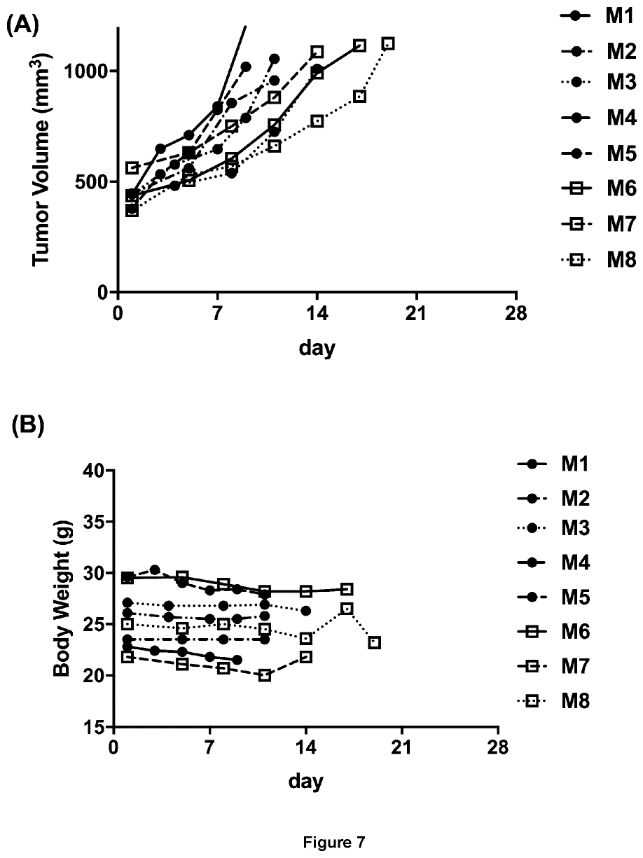

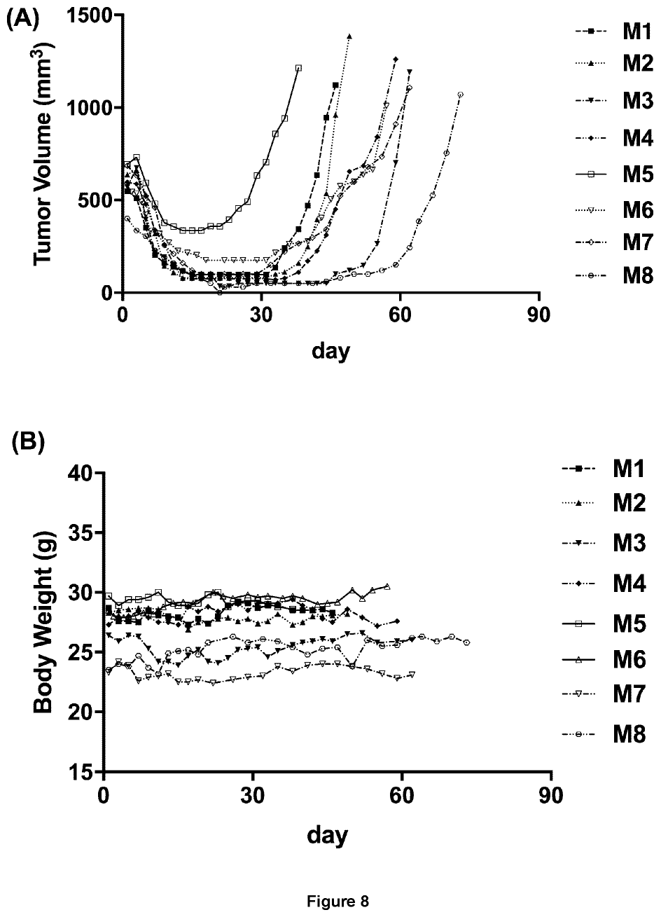

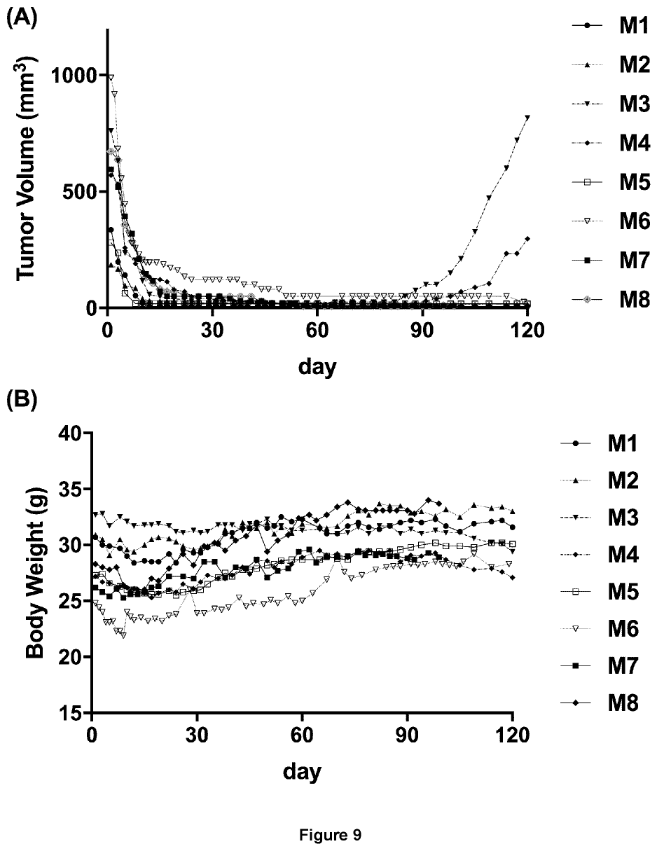

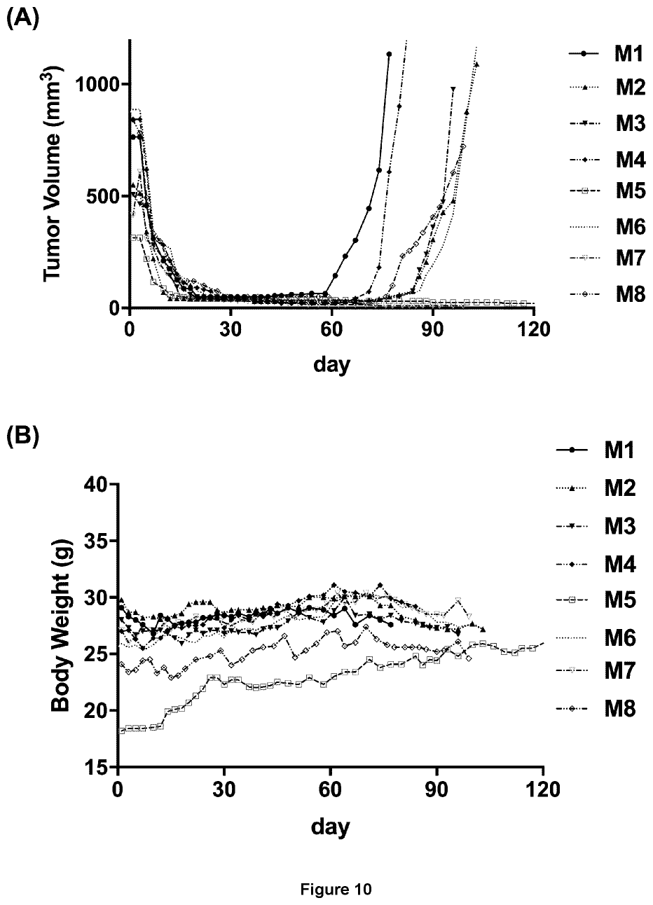

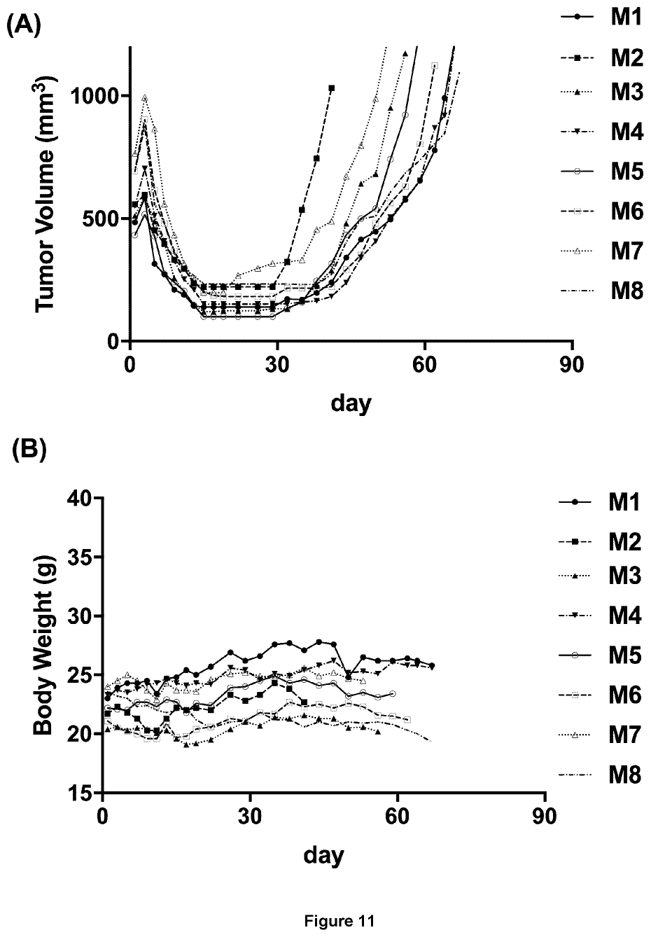

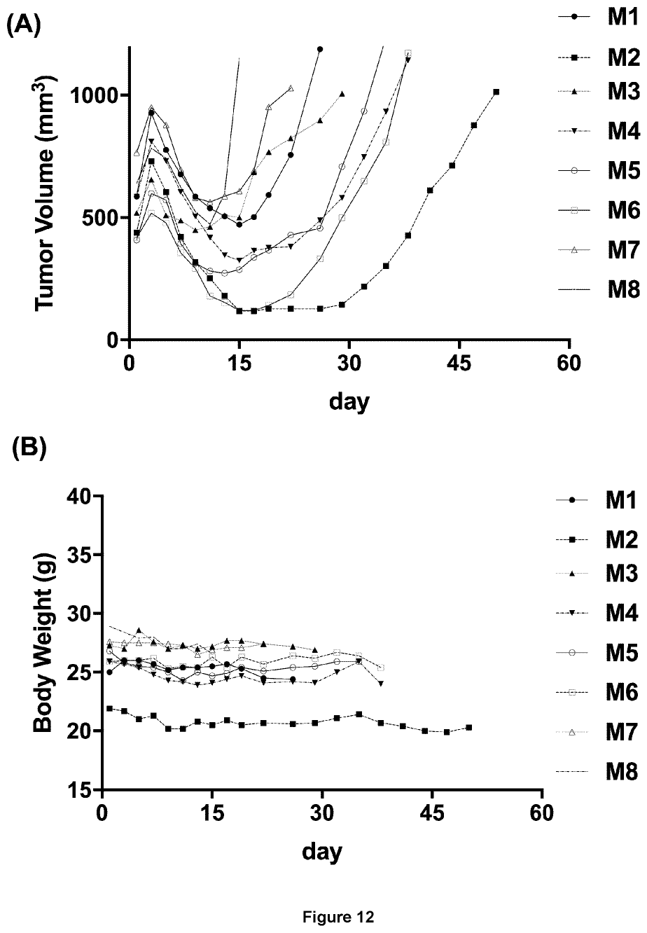

The results of the endoradiotherapy study are shown in Table 6 and FIG. 6, and the changes of LNCaP tumor volume and mouse body weight over time after treatment are shown in FIGS. 7-12. The tumor volume of the control group (Group A in Table 6, FIG. 7(A)) increased continuously after treatment (saline injection), and the median survival of the control group was only 14 days (mice were euthanized when their tumor volume reached 1000 mm³). The tumors in mice treated with ¹⁷⁷Lu-PSMA-617 (18.5 MBq, Group B in Table 6, FIG. 8A) shrank initially but grew back later, leading to an improved median survival of 58 days. The changes in tumor size over time for the mice treated with ¹⁷⁷Lu-HTK01169 (Groups C-F in Table 6, FIGS. 9(A)-12(A)) depended on the injected radioactivity with higher radioactivity leading to more effective and prolonged tumor growth inhibition. The median survivals for the groups of mice treated with 18.5, 9.3, 4.6, and 2.3 MBq of ¹⁷⁷Lu-HTK01169 were >120, 103, 61 and 28 days, respectively. No weight loss was observed for all mice regardless of their treatment (FIGS. 7(B)-12(B)), and all mice treated with 18.5 MBq of ¹⁷⁷Lu-HTK01169 (Group C in Table 6) survived until the end of the study (Day 120).

TABLE 6
Data of the radiotherapy study, including median survival after treatment of
tumors with saline, ¹⁷⁷Lu-PSMA-617 or ¹⁷⁷Lu-HTK01169.

| | Treatment | Injected radioactivity (MBq) | | Tumor volume (mm³) | Median |
|---|---|---|---|---|---|
| Group | (n = 8) | Theoretical | Measured (mean ± SD) | Day 0 (mean ± SD) | survival (Day) |
| A | Saline | | | 440 ± 59 | 14 |
| B | ¹⁷⁷Lu-PSMA-617 | 18.5 | 18.9 ± 0.9 | 589 ± 93 | 58 |
| C | ¹⁷⁷Lu-HTK01169 | 18.5 | 18.8 ± 1.6 | 531 ± 239 | >120 |
| D | ¹⁷⁷Lu-HTK01169 | 9.3 | 9.7 ± 0.3 | 640 ± 221 | 103 |
| E | ¹⁷⁷Lu-HTK01169 | 4.6 | 4.5 ± 0.2 | 586 ± 117 | 61 |
| F | ¹⁷⁷Lu-HTK01169 | 2.3 | 2.3 ± 0.1 | 545 ± 124 | 28 |

1.3 Discussion

The use of small-molecule albumin binders to extend the circulation time of pharmaceuticals and maximize their tumor uptake has become an attractive strategy for the design of endoradiotherapeutic agents. The pioneering work was conducted mainly by ETH Zurich scientists using a D-Lys acylated at the ε-amino group with a 4-(p-iodophenyl)butyric acid as the albumin-binding motif.²² Previous studies focused on applying this strategy for the design of folate-receptor-targeted radiopharmaceuticals.²³ As folate receptor a and proton-coupled folate transporter are highly expressed in renal proximal tubules, radiolabeled folate derivatives generally result in very high and sustained kidney uptake.²³ Radiolabeled folate derivatives with a built-in albumin binder were reported to significantly extend blood retention time, increase tumor uptake and improve tumor-to-kidney uptake ratios.²³

Recently, attempts were also made to use this strategy for the design of PSMA-targeted endoradiotherapeutic agents with albumin-binding motifs.²⁴⁻²⁸ Among the reported albumin-conjugated PSMA-targeted agents, ¹⁷⁷Lu-PSMA-ALB-02, ¹⁷⁷Lu-PSMA-ALB-056 and ¹⁷⁷Lu-RPS-063 were shown to deliver around 1.8-, 2.3- and 3.8-fold higher radiation dose than ¹⁷⁷Lu-PSMA-617 to PSMA-expressing tumors.²⁶⁻²⁸ In addition, ¹⁷⁷Lu-PSMA-ALB-056 was further evaluated in an endoradiotherapy study in mice bearing PSMA-expressing PC-3 PIP tumors.²⁷ The mice treated with ¹⁷⁷Lu-PSMA-617 or 177Lu-PSMA-ALB-056 showed extended median survival when compared with the mice in the control group treated with saline. Most importantly, using only 2 MBq of ¹⁷⁷Lu-PSMA-ALB-056 was able to produce slightly better median survival when compared to that from using 5 MBq of ¹⁷⁷Lu-PSMA-617 (36 vs 32 days).

In this Example, the conjugation of a novel albumin binder was used to further improve tumor uptake of ¹⁷⁷Lu-PSMA-617, the most studied PSMA-targeted endoradiotherapeutic agent. The most common albumin-binding motif reported in literature consisted of a D-Lys that is acylated by 4-(p-iodophenyl)butyric acid at the ε-amino group.²²⁻²³ Since the α-carboxylic group of D-Lys is part of albumin-binding motif, it cannot be used for conjugation to the peptide via solid phase synthesis.²⁹ As shown in the structure of Lu-HTK01169, a Glu residue is used in place of D-Lys. As a result, the carboxylic group at the Glu side chain can be used for binding to albumin, and the α-carboxylic group was used for conjugation to the peptide via solid phase synthesis. As shown in this Example, modification of the linker between the DOTA chelator and the PSMA-targeting Lys-urea-Glu did not adversely affect therapeutic efficacy, which confirms reports that such linker modifications can be well tolerated.¹⁷ In fact, as shown in this Example, Lu-HTK01169 was observed to have a 6-fold improvement in PSMA binding compared to Lu-PSMA-617 (Ki values: 0.04±0.01 vs 0.24±0.06 nM). Without wishing to be bound by theory, the improved PSMA binding may be due to the introduction of the highly lipophilic 4-(p-iodophenyl)butyryl group.

The ability of ¹⁷⁷Lu-HTK01169 to bind albumin was assessed by plasma protein binding assay. In contrast to the ˜17% of free ¹⁷⁷Lu-PSMA-617, only <1% of ¹⁷⁷Lu-HTK01169 was observed under the same conditions, demonstrating the capability of the albumin binder modified derivative to interact with plasma proteins.

The addition of an albumin binder to extend the blood retention time and maximize tumor uptake were confirmed by SPECT/CT and biodistribution studies. ¹⁷⁷Lu-HTK01169 not only showed improved peaked tumor uptake (¹⁷⁷Lu-HTK01169: 55.9±12.5% ID/g; ¹⁷⁷Lu-PSMA-617: 15.1±5.58% ID/g), but most importantly the uptake was sustained, rather than decreasing over time like ¹⁷⁷Lu-PSMA-617. Without wishing to be bound by theory, this could be due to, in part, the improved PSMA binding of Lu-HTK01169 over Lu-PSMA-617. Compared with ¹⁷⁷Lu-PSMA-617, improved uptake combined with longer residence time provided an 8.3-fold higher radiation dose of 1⁷⁷Lu-PSMA-617 to LNCaP tumor xenografts. Such design strategy may be even more significant for radioisotopes with a longer half-life such as the α-emitter ²²⁵Ac (t_{1/2}: ²²⁵Ac, 9.95 d; ¹⁷⁷Lu, 6.65 d). Currently the clinically used ²²⁵Ac is extracted from ²²⁹Th, and is in limited supply.³⁰⁻³¹ Switching from ²²⁵Ac-PSMA-617 to ²²⁵Ac-HTK01169 may significantly increase the number of patients who can be treated with ²²⁵Ac-labeled PSMA-targeting radioligands.

This example showed a quick reduction in size of LNCaP tumor xenografts over time with the injection of ˜37 MBq of either ¹⁷⁷Lu-PSMA-617 or ¹⁷⁷Lu-HTK01169 (FIG. 2). The ˜37 MBq injected radioactivity used for the acquisition of high-resolution SPECT images could have exceeded the dose of ¹⁷⁷Lu-HTK01169 needed to treat LNCaP tumors. Therefore, the endoradiotherapy study in this Example compared the median survivals of mice treated with 18.5 MBq of ¹⁷⁷Lu-PSMA-617 or ¹⁷⁷Lu-HTK01169, as well as with only one half (9.3 MBq), one quarter (4.6 MBq) or one-eighth (2.3 MBq) dose of ¹⁷⁷Lu-HTK01169. The one-eighth dose (2.3 MBq) of ¹⁷⁷Lu-HTK01169 did not produce similar median survival when compared to that of ¹⁷⁷Lu-HTK01169 (18.5 MBq, Table 6) as predicted from the dosimetry data. However, it was observed that the median survival of mice treated with one quarter dose (4.5 MBq) of ¹⁷⁷Lu-HTK01169 was slightly better than that of mice treated with 18.5 MBq of ¹⁷⁷Lu-PSMA-617 (61 vs 58 days, Table 6).

Among the reported albumin-binder-conjugated PSMA-targeted endoradiotherapeutic agents, only ¹⁷⁷Lu-PSMA-ALB-056 has been evaluated in a radiotherapy study and compared directly with ¹⁷⁷Lu-PSMA-617.²⁷ There are two main differences between the findings of this Example and those reported for ¹⁷⁷Lu-PSMA-ALB-056, reported by Umbricht et al.²⁷ For the tumor model, this Example used LNCaP, an unmodified endogenous prostate cancer cell line. The evaluation of ¹⁷⁷Lu-PSMA-ALB-056 used PC-3 PIP, a transduced cell line with a much higher PSMA expression level than LNCaP cells.²⁷ Consequently, the treatment doses (2 and 5 MBq) of ¹⁷⁷Lu-PSMA-ALB-056 and ¹⁷⁷Lu-PSMA-617 in the previously reported study were lower than those used in this Example (2.3-18.5 MBq). The second difference is the size of tumors. Unlike the ˜100 mm³ average tumor size used to evaluate ¹⁷⁷Lu-PSMA-ALB-056, the range of tumor sizes in the present Example when treatment began with ¹⁷⁷Lu-PSMA-617 or ¹⁷⁷Lu-HTK01169 was 531-640 mm³. The larger tumors in this Example likely conferred a higher degree of resistance to the treatment, and subsequently required a higher radiation dose to achieve the similar growth inhibition.

Compared to ¹⁷⁷Lu-PSMA-617, the albumin-binder-conjugated ¹⁷⁷Lu-HTK01169 delivered 3.7-fold higher peak uptake and 8.3-fold overall radiation dose to LNCaP tumor xenografts. The endoradiotherapy study in LNCaP tumor-bearing mice also showed that only a quarter of the administered activity of ¹⁷⁷Lu-PSMA-617 is needed for ¹⁷⁷Lu-HTK01169 to achieve similar treatment efficacy. When translated to the clinic, HTK01169 radiolabeled with ¹⁷⁷Lu or ²²⁵Ac could potentially also produce similar or improved radiotherapeutic efficacy with only a fraction of administered activity of ¹⁷⁷Lu-PSMA-617. The newly introduced albumin binder in HTK01169 can be constructed directly on solid phase along peptide elongation. Based on promising data obtained from ¹⁷⁷Lu-HTK01169, this new albumin-binding motif could potentially be applied to other (radio)peptides to extend their blood retention times and maximize therapeutic efficacy.

### Example 2: Modified Metal-Chelating PSMA-Binding Compounds

2.1 Materials and Methods

2.11 General Methods

All chemicals and solvents were obtained from commercial sources, and used without further purification. PSMA-targeted peptides were synthesized using solid phase approach on an AAPPTec (Louisville, KY) Endeavor 90 peptide synthesizer. Purification and quality control of cold and radiolabeled peptides were performed on Agilent HPLC systems equipped with a model 1200 quaternary pump, a model 1200 UV absorbance detector (set at 220 nm), and a Bioscan (Washington, DC) NaI scintillation detector. The operation of Agilent HPLC systems was controlled using the Agilent ChemStation software. The HPLC columns used were a semi-preparative column (Luna C18, 5μ, 250×10 mm) and an analytical column (Luna C18, 5μ, 250×4.6 mm) purchased from Phenomenex (Torrance, CA). The collected HPLC eluates containing the desired peptide were lyophilized using a Labconco (Kansas City, MO) FreeZone 4.5 Plus freeze-drier. Mass analyses were performed using an AB SCIEX (Framingham, MA) 4000 QTRAP mass spectrometer system with an ESI ion source. C18 Sep-Pak cartridges (1 cm³, 50 mg) were obtained from Waters (Milford, MA). ⁶⁸Ga was eluted from an iThemba Labs (Somerset West, South Africa) generator, and was purified using a DGA resin column from Eichrom Technologies LLC (Lisle, IL). Radioactivity of ⁶⁸Ga-labeled peptides was measured using a Capintec (Ramsey, NJ) CRC®-25R/W dose calibrator, and the radioactivity of mouse tissues collected from biodistribution studies were counted using a Perkin Elmer (Waltham, MA) Wizard2 2480 automatic gamma counter.

2.12 Synthesis of HTK03026, HTK03027, HTK03029, and HTK03041

The structures of HTK03026, HTK03027, HTK03029, and HTK03041 are shown below:

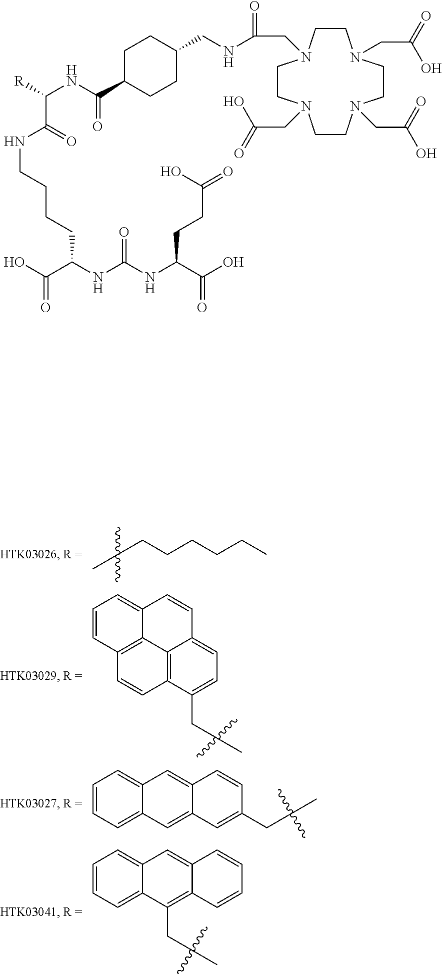

Solid-phase synthesis of HTK3026, HTK03027, HTK03029 and HTK03041 was modified from literature procedures.¹⁶ Fmoc-Lys(ivDde)-Wang resin (0.3 mmol, 0.61 mmol/g loading) was suspended in DMF for 30 min. Fmoc was then removed by treating the resin with 20% piperidine in DMF (3×8 min). The isocyanate derivative of di-t-butyl ester of glutamate (3 eq.) was prepared according to literature procedures,¹⁷ and added to the lysine-immobilized resin and reacted for 16 h. Afterwashing the resin with DMF, the ivDde-protecting group was removed with 2% hydrazine in DMF (5×5 min). Fmoc-2-Aoc-OH (for HTK03026), Fmoc-Ala(2-Anth)-OH (for HTK03027), Fmoc-Ala(1-Pyrenyl)-OH (for HTK03029) or Fmoc-Ala(9-Anth)-OH (for HTK03041) was then coupled to the side chain of Lys using Fmoc-protected amino acid (3 eq.), HBTU (3 eq.), HOBT (3 eq.) and N,N-diisopropylethylamine (8 eq.). Afterwards, elongation was continued with the addition of Fmoc-tranexamic acid, and finally DOTA-tris(t-bu)ester (2-(4,7,10-tris(2-(t-butoxy)-2-oxoehtyl)-1,4,7,10)-tetraazacyclododecan-1-yl)acetic acid).

The peptide was then deprotected and simultaneously cleaved from the resin by treating with 95/5 trifluoroacetic acid (TFA)/triisopropylsilane (TIS) for 2 h at room temperature. After filtration, the peptide was precipitated by the addition of cold diethyl ether to the TFA solution. The crude peptide was purified by HPLC using the semi-preparative column. The eluates containing the desired peptide were collected, pooled, and lyophilized. For HTK03026, the HPLC conditions were 27% acetonitrile in water with 0.1% TFA at a flow rate of 4.5 mL/min. The retention time was 10.7 min. ESI-MS: calculated [M+H]⁺ for HTK03026 C₄₅H₇₅N₉O₁₆ 986.5; found [M+H]⁺ 986.6. For HTK03027, the HPLC conditions were 32% acetonitrile in water with 0.1% TFA at a flow rate of 4.5 mL/min. The retention time was 7.1 min. ESI-MS: calculated [M+H]⁺ for HTK03027 C₅₃H₇₄N₉O₁₆ 1092.5; found [M+H]⁺ 1094.6. For HTK03029, the HPLC conditions were 33% acetonitrile in water with 0.1% TFA at a flow rate of 4.5 mL/min. The retention time was 7.3 min. ESI-MS: calculated [M+H]⁺ for HTK03029 C₅₅H₇₄N₉O₁₆ 1116.5; found [M+H]⁺ 1116.6. For HTK03041, the HPLC conditions were 31% acetonitrile in water with 0.1% TFA at a flow rate of 4.5 mL/min. The retention time was 7.2 min. ESI-MS: calculated [M+H]⁺ for HTK03041 C₅₃H₇₄N₉O₁₆ 1092.5; found [M+H]⁺ 1092.6.

2.13 Synthesis of HTK03024, HTK03055, HTK03056, HTK03058, HTK03082, HTK03085, HTK03086, HTK03087, HTK03089, and HTK03090

The structures of HTK03024, HTK03055, HTK03056, HTK03058, HTK03085, HTK03086, HTK03087, HTK03089, and HTK03090 are shown below:

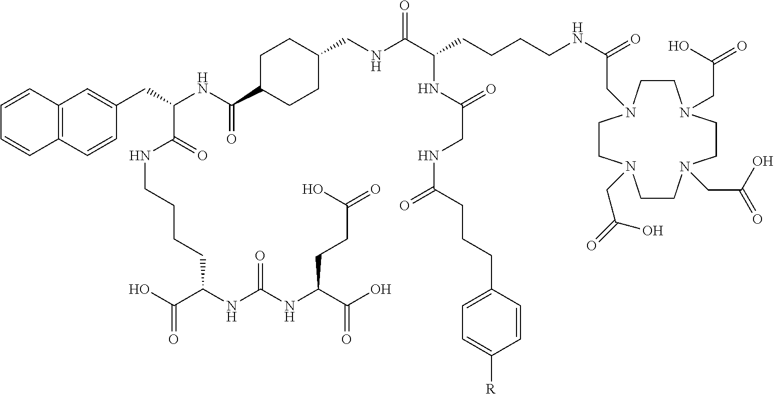

wherein R═I (HTK03024), Cl (HTK03055), H (HTK03056), Br (HTK03058), F (HTK03085), OCH₃ (HTK03086), NH₂ (HTK03087), NO₂ (HTK03089), or CH₃ (HTK03090).

The structure of HTK03082 is shown below:

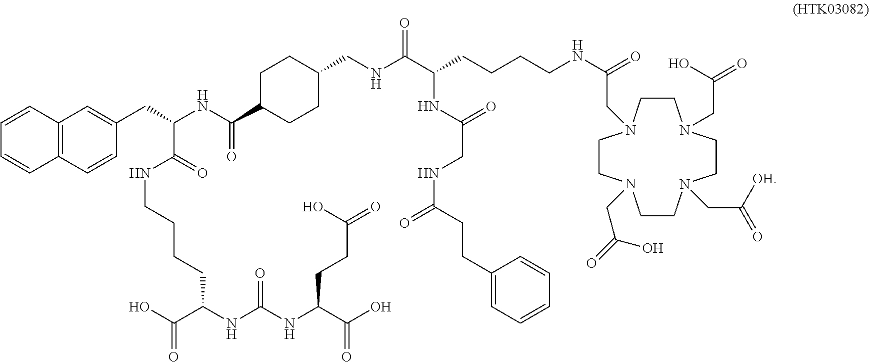

Fmoc-Lys(ivDde)-Wang resin (0.3 mmol, 0.61 mmol/g loading) was suspended in DMF for 30 min. Fmoc was then removed by treating the resin with 20% piperidine in DMF (3×8 min). The isocyanate derivative of di-t-butyl ester of glutamate (3 eq.) was prepared according to literature procedures,¹⁷ and added to the lysine-immobilized resin and reacted for 16 h. After washing the resin with DMF, the ivDde-protecting group was removed with 2% hydrazine in DMF (5×5 min). Fmoc-2-NaI—OH was then coupled to the side chain of Lys followed by Fmoc-tranexamic acid, Fmoc-Lys(ivDde)-OH, and Fmoc-Gly-OH via solid-phase peptide synthesis using Fmoc-based chemistry. All couplings were carried out in DMF using Fmoc-protected amino acid (3 eq.), HBTU (3 eq.), HOBT (3 eq.), and DIEA (8 eq.). Afterwards, elongation was continued with the addition of 4-(p-iodophenyl)butyric acid (for HTK03024), 4-(p-chlorophenyl)butyric acid (for HTK03055), 4-phenylbutyric acid (for HTK03056), 4-(p-bromophenyl)butyric acid (for HTK03058), 3-phenylpropanoic acid (for HTK03082), 4-(p-fluorophenyl)butyric acid (for HTK03085), 4-(p-methoxyphenyl)butyric acid (for HTK03086), 4-(p-(t-butyloxycarbonyl)aminophenyl)butyric acid (for HTK03087), 4-(p-nitrophenyl)butyric acid (for HTK03089), or 4-(p-tolyl)butyric acid (for HTK03090) were coupled to the same peptide-bound resin using Fmoc-based chemistry. After selective removal of the ivDde-protecting group with 2% hydrazine in DMF (5×5 min), the chelator DOTA was then coupled to the side chain of Lys to give the precursors.

The peptide was then deprotected and simultaneously cleaved from the resin by treating with 95/5 trifluoroacetic acid (TFA)/triisopropylsilane (TIS) for 2 h at room temperature. After filtration, the peptide was precipitated by the addition of cold diethyl ether to the TFA solution. The crude peptide was purified by HPLC using the semi-preparative column. The eluates containing the desired peptide were collected, pooled, and lyophilized. For HTK03024, the HPLC conditions were 37% acetonitrile in water with 0.1% TFA at a flow rate of 4.5 mL/min. The retention time was 8.8 min. ESI-MS: calculated [M+H]⁺ for HTK03024 C₆₇H₉₆N₁₂O₁₉I 1499.6; found [M+H]⁺ 1499.6. For HTK03055, the HPLC conditions were 35% acetonitrile in water with 0.1% TFA at a flow rate of 4.5 mL/min. The retention time was 9.7 min. ESI-MS: calculated [M+H]⁺ for HTK03055 C₆₇H₉₆N₁₂O₁₉Cl 1407.7; found [M+H]⁺ 1407.7. For HTK03056, the HPLC conditions were 0-80% acetonitrile in water with 0.1% TFA at a flow rate of 4.5 mL/min in 20 min. The retention time was 13.4 min. ESI-MS: calculated [M+H]⁺ for HTK03056 C₆₇H₉₇N₁₂O₁₉ 1373.7; found [M+H]⁺ 1373.8. For HTK03058, the HPLC conditions were 0-80% acetonitrile in water with 0.1% TFA at a flow rate of 4.5 mL/min in 20 min. The retention time was 13.4 min. ESI-MS: calculated [M+H]⁺ for HTK03058 C₆₇H₉₆N₁₂O₁₉Br 1451.6; found [M+H]⁺ 1451.6. For HTK03082, the HPLC conditions were 31% acetonitrile in water with 0.1% TFA at a flow rate of 4.5 mL/min. The retention time was 11.1 min. ESI-MS: calculated [M+H]⁺ for HTK03082 C₆₆H₉₅N₁₂O₁₉ 1359.7; found [M+H]⁺ 1359.9. For HTK03085, the HPLC conditions were 34% acetonitrile in water with 0.1% TFA at a flow rate of 4.5 mL/min. The retention time was 9.0 min. ESI-MS: calculated [M+H]⁺ for HTK03085 C₆₇H₉₆N₁₂O₁₉F 1391.7; found [M+H]⁺ 1391.9. For HTK03086, the HPLC conditions were 33% acetonitrile in water with 0.1% TFA at a flow rate of 4.5 mL/min. The retention time was 9.1 min. ESI-MS: calculated [M+H]⁺ for HTK03086 C₆₈H₉N₁₂O₂₀ 1403.7; found [M+H]⁺ 1404.1. For HTK03087, the HPLC conditions were 23% acetonitrile in water with 0.1% TFA at a flow rate of 4.5 mL/min. The retention time was 13.9 min. ESI-MS: calculated [M+H]⁺ for HTK03087 C₆₇H₉₈N₁₃O₁₉ 1388.7; found [M+H]⁺ 1389.0. For HTK03089, the HPLC conditions were 33% acetonitrile in water with 0.1% TFA at a flow rate of 4.5 mL/min. The retention time was 10.6 min. ESI-MS: calculated [M+H]⁺ for HTK03089 C₆₇H₉₆N₁₃O₂₁ 1418.7; found [M+H]⁺ 1419.0. For HTK03090, the HPLC conditions were 35% acetonitrile in water with 0.1% TFA at a flow rate of 4.5 mL/min. The retention time was 9.1 min. ESI-MS: calculated [M+H]⁺ for HTK03090 C₆₈H₉₉N₁₂O₁₉ 1387.7; found [M+H]⁺ 1387.9.

2.14 Synthesis of Ga-Labeled Standards

To prepare Ga-labeled standards, a solution of each precursor was incubated with GaCl₃ (5 eq.) in NaOAc buffer (0.1 M, 500 μL, pH 4.2) at 80° C. for 15 min. The reaction mixture was then purified by HPLC using the semi-preparative column, and the HPLC eluates containing the desired peptide were collected, pooled, and lyophilized. For Ga-HTK03026, the HPLC conditions were 27% acetonitrile in water with 0.1% TFA at a flow rate of 4.5 mL/min. The retention time was 9.4 min. ESI-MS: calculated [M+H]⁺ for Ga-HTK03026 C₄₄H₇₃N₉O₁₆Ga 1052.4; found [M+H]⁺ 1052.5. For Ga-HTK03027, the HPLC conditions were 32% acetonitrile in water with 0.1% TFA at a flow rate of 4.5 mL/min. The retention time was 9.5 min. ESI-MS: calculated [M+H]⁺ for Ga-HTK03027 C₅₃H₇₂N₉O₁₆Ga 1159.4; found [M+H]⁺ 1161.4. For HTK03029, the HPLC conditions were 33% acetonitrile in water with 0.1% TFA at a flow rate of 4.5 mL/min. The retention time was 10.3 min. ESI-MS: calculated [M+H]⁺ for Ga-HTK03029 C₅₅H₇₂N₉O₁₆Ga 1183.4; found [M+H]⁺ 1183.4. For Ga-HTK03041, the HPLC conditions were 31% acetonitrile in water with 0.1% TFA at a flow rate of 4.5 mL/min. The retention time was 9.3 min. ESI-MS: calculated [M+H]⁺ for Ga-HTK03041 C₅₃H₇₂N₉O₁₆Ga 1159.4; found [M+H]⁺ 1159.4. For Ga-HTK03024, the HPLC conditions were 39% acetonitrile in water with 0.1% TFA at a flow rate of 4.5 mL/min. The retention time was 8.0 min. ESI-MS: calculated [M+H]⁺ for Ga-HTK03024 Ce₇H₉₃N₁₂O₁₉Ga 1565.5; found [M+H]⁺ 1565.5. For Ga-HTK03055, the HPLC conditions were 35% acetonitrile in water with 0.1% TFA at a flow rate of 4.5 mL/min. The retention time was 12.7 min. ESI-MS: calculated [M+H]⁺ for Ga-HTK03055 C₆₇H₉₄N₁₂O₁₉ClGa 1474.6; found [M+H]²+738.4. For Ga-HTK03056, the HPLC conditions were 34% acetonitrile in water with 0.1% TFA at a flow rate of 4.5 mL/min. The retention time was 9.0 min. ESI-MS: calculated [M+H]⁺ for Ga-HTK03056 Ce₇H₉₄N₁₂O₁₉Ga 1439.6; found [M+H]⁺ 1439.8. For Ga-HTK03058, the HPLC conditions were 34% acetonitrile in water with 0.1% TFA at a flow rate of 4.5 mL/min. The retention time was 10.3 min. ESI-MS: calculated [M+H]⁺ for Ga-HTK03058 Ce₇H₉₃N₁₂O₁₉BrGa 1517.5; found [M+H]⁺ 1518.0. For Ga-HTK03082, the HPLC conditions were 31% acetonitrile in water with 0.1% TFA at a flow rate of 4.5 mL/min. The retention time was 12.5 min. ESI-MS: calculated [M+H]⁺ for Ga-HTK03082 C₆₆H₉₃N₁₂O₁₉Ga 1426.6; found [M+H]⁺ 1426.9. For Ga-HTK03085, the HPLC conditions were 34% acetonitrile in water with 0.1% TFA at a flow rate of 4.5 mL/min. The retention time was 9.0 min. ESI-MS: calculated [M+H]⁺ for Ga-HTK03085 Ce₇H₉₄N₁₂O₁₉FGa 1458.6; found [M+H]⁺ 1459.6. For Ga-HTK03086, the HPLC conditions were 33% acetonitrile in water with 0.1% TFA at a flow rate of 4.5 mL/min. The retention time was 10.7 min. ESI-MS: calculated [M+H]⁺ for Ga-HTK03086 C₆₃H₉₆N₁₂O₂₀Ga 1469.6; found [M+H]⁺ 1469.8. For Ga-HTK03087, the HPLC conditions were 23% acetonitrile in water with 0.1% TFA at a flow rate of 4.5 mL/min. The retention time was 14.7 min. ESI-MS: calculated [M+H]⁺ for Ga-HTK03087 C₆₇H₉₆N₁₃O₁₉Ga 1455.6; found [M+H]⁺ 1455.8. For Ga-HTK03089, the HPLC conditions were 33% acetonitrile in water with 0.1% TFA at a flow rate of 4.5 mL/min. The retention time was 12.0 min. ESI-MS: calculated [M+H]⁺ for Ga-HTK03089 C₆₇H₉₄N₁₃O₂₁Ga 1485.6; found [M+H]⁺ 1485.9. For Ga-HTK03090, the HPLC conditions were 35% acetonitrile in water with 0.1% TFA at a flow rate of 4.5 mL/min. The retention time was 11.3 min. ESI-MS: calculated [M+H]⁺ for Ga-HTK03090 C₆₈H₉₇N₁₂O₁₉Ga, 1454.6; found [M+H]⁺ 1455.8.

2.15 Cell Culture

LNCap cell line was obtained from ATCC (LNCaP clone FGC, CRL-1740). It was established from a metastatic site of left supraclavicular lymph node of human prostatic adenocarcinoma. Cells were cultured in PRMI 1640 medium supplemented with 10% FBS, penicillin (100 U/mL) and streptomycin (100 μg/mL) at 37° C. in a humidified incubator containing 5% CO₂. Cells grown to 80-90% confluence were then washed with sterile phosphate-buffered saline (1×PBS pH 7.4) and trypsinization. The collected cells number was counted with a Hausser Scientific (Horsham, PA) Hemacytometer.

2.16 Synthesis of ⁶⁸Ga-Labeled Compounds

Purified ⁶⁸Ga in 0.5 mL of water was added into a 4 mL glass vial preloaded with 0.7 mL of HEPES buffer (2 M, pH 5.0) and 50 μg of DOTA-containing precursor. The radiolabeling reaction was carried out under microwave heating for 1 min. The reaction mixture was purified by HPLC using the same semipreparative column and conditions provided in Section 2.14 for the purification of their respective nonradioactive Ga-labeled standards.

2.17 PET/CT Imaging and Biodistribution

Imaging and biodistribution experiments were performed using NODSCID 1L2RγKO male mice. Mice were anesthetized by inhalation with 2% isoflurane in oxygen, and implanted subcutaneously with 1×10⁷ LNCaP cells behind left shoulder. Mice were imaged or used in biodistribution studies when the tumor grew up to reach 5-8 mm in diameter during 5-6 weeks.

PET imaging experiments were conducted using Siemens Inveon micro PET/CT scanner. Each tumor bearing mouse was injected 6-8 MBq of 68Ga-labeled tracer through the tail vein under anesthesia (2% isoflurane in oxygen). The mice were allowed to recover and roam freely in their cage. After 50 min, the mice were sedated again with 2% isoflurane in oxygen inhalation and positioned in the scanner. A 10-min CT scan was conducted first for localization and attenuation correction after segmentation for reconstructing the PET images. Then, a 10-min static PET imaging was performed to determined uptake in tumor and other organs. The mice were kept warm by a heating pad during acquisition. For imaging studies acquired at 3 h post-injection (p.i.), the mice were placed in the micro PET/CT scanner at 170 min p.i. Then, the CT acquisitions were conducted as described above, a 15-min static PET imaging was performed to determined uptake in tumor and other organs.

For biodistribution studies, the mice were injected with the radiotracer as described above. At predetermined time points (1 or 3 h), the mice was anesthetized with 2% isoflurane inhalation, and euthanized by CO₂ inhalation. Blood was withdrawn immediately from the heart, and the organs/tissues of interest were collected. The collected organs/tissues were weighed and counted using an automatic gamma counter. The uptake in each organ/tissue was normalized to the injected dose using a standard curve, and expressed as the percentage of the injected dose per gram of tissue (% ID/g).

2.2 Results

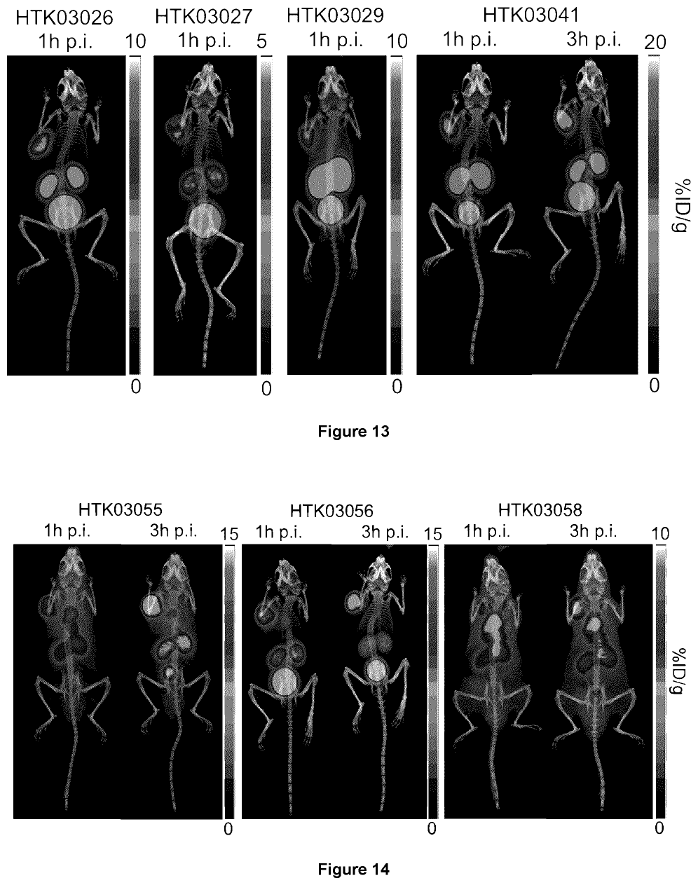

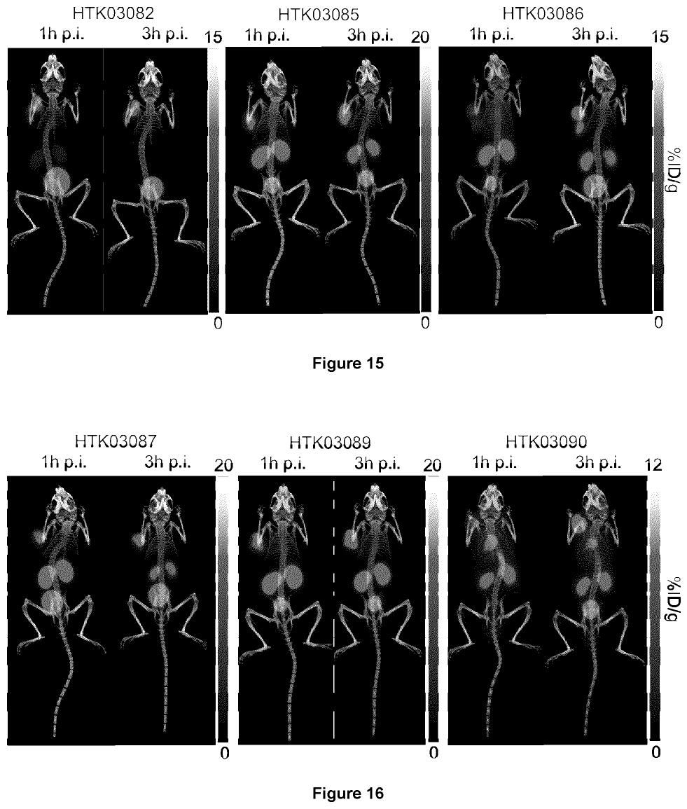

Results for this Example are shown in Tables 7-10 and FIGS. 13-16. In combination with the results of Example 1, these results show that the various compounds encompassed within Formula 1-a and Formula 1-b would be particularly useful.

TABLE 7
Biodistribution data and tumor-to-background contrast ratios of ⁶⁸Ga-labeled
HTK03026, HTK03027, HTK03029 and HTK03041 in mice bearing
PSMA-expressing LNCAP cancer xenografts.

| | ⁶⁸Ga- | ⁶⁸Ga- | ⁶⁸Ga- | | |
|---|---|---|---|---|---|
| | HTK03026 | HTK03027 | HTK03029 | ⁶⁸Ga-HTK03041 | |
| Tissue | 1 h | 1 h | 1 h | 1 h | 3 h |
| (% ID/g) | (n = 7) | (n = 5) | (n = 5) | (n = 6) | (n = 6) |
| Blood | 0.70 ± 0.19 | 1.56 ± 0.57 | 3.93 ± 0.91 | 1.43 ± 0.30 | 0.83 ± 0.27 |
| Urine | 575 ± 245 | 844 ± 645 | 175 ± 178 | 173 ± 93.01 | 171 ± 93.3 |
| Fat | 0.45 ± 0.11 | 1.03 ± 0.67 | 1.57 ± 0.42 | 2.06 ± 0.59 | 1.06 ± 0.20 |
| Seminal | 0.43 ± 0.31 | 10.6 ± 10.9 | 6.74 ± 10.7 | 1.03 ± 0.27 | 4.56 ± 9.00 |
| Testes | 0.35 ± 0.22 | 0.56 ± 0.18 | 1.02 ± 0.13 | 1.34 ± 0.22 | 0.89 ± 0.12 |
| Intestine | 0.29 ± 0.06 | 0.44 ± 0.12 | 0.77 ± 0.11 | 1.14 ± 0.18 | 0.67 ± 0.09 |
| Stomach | 0.10 ± 0.03 | 0.17 ± 0.10 | 0.31 ± 0.08 | 0.41 ± 0.11 | 0.26 ± 0.06 |
| Spleen | 1.34 ± 0.54 | 3.36 ± 2.54 | 7.08 ± 5.08 | 8.95 ± 3.22 | 4.19 ± 1.58 |
| Liver | 0.24 ± 0.06 | 0.46 ± 0.10 | 1.32 ± 0.63 | 1.38 ± 0.25 | 0.82 ± 0.15 |
| Pancreas | 0.71 ± 1.04 | 0.57 ± 0.20 | 0.94 ± 0.18 | 1.47 ± 0.16 | 0.71 ± 0.12 |
| Adrenal glands | 1.84 ± 0.95 | 3.55 ± 2.33 | 4.91 ± 1.57 | 8.90 ± 2.56 | 4.04 ± 1.20 |
| Kidneys | 68.5 ± 28.2 | 85.6 ± 73.5 | 198 ± 49.3 | 170 ± 26.4 | 121 ± 37.6 |
| Lungs | 0.82 ± 0.26 | 1.66 ± 0.67 | 3.59 ± 0.69 | 4.32 ± 0.62 | 2.14 ± 0.39 |
| Heart | 0.30 ± 0.06 | 0.56 ± 0.24 | 1.25 ± 0.27 | 1.82 ± 0.21 | 0.93 ± 0.17 |
| Tumor | 12.5 ± 2.90 | 13.3 ± 5.44 | 13.9 ± 6.58 | 23.1 ± 6.11 | 28.2 ± 9.17 |
| Muscle | 0.28 ± 0.13 | 0.48 ± 0.37 | 0.63 ± 0.10 | 0.75 ± 0.09 | 0.40 ± 0.06 |
| Bone | 0.54 ± 0.20 | 0.70 ± 0.33 | 0.92 ± 0.14 | 1.29 ± 0.45 | 0.98 ± 0.40 |
| Brain | 0.06 ± 0.02 | 0.06 ± 0.03 | 0.09 ± 0.02 | 0.10 ± 0.05 | 0.06 ± 0.02 |
| Tail | 0.90 ± 0.56 | 0.98 ± 0.38 | 2.18 ± 0.65 | 1.36 ± 0.45 | 0.86 ± 0.73 |
| Thyroid | 0.35 ± 0.08 | 0.76 ± 0.33 | 1.57 ± 0.32 | 2.48 ± 0.44 | 1.19 ± 0.23 |
| Salivary | 0.83 ± 0.22 | 1.86 ± 0.91 | 3.27 ± 1.02 | 4.99 ± 0.88 | 2.11 ± 0.57 |
| Lacrimal | 1.33 ± 0.90 | 1.00 ± 0.56 | 1.06 ± 0.49 | 2.44 ± 1.79 | 2.82 ± 3.84 |
| Tumor:Blood | 18.6 ± 5.54 | 8.57 ± 2.83 | 3.39 ± 1.01 | 17.3 ± 7.24 | 36.6 ± 15.4 |
| Tumor:Muscle | 52.8 ± 24.9 | 35.0 ± 18.7 | 21.3 ± 7.50 | 31.6 ± 12.1 | 73.8 ± 30.9 |
| Tumor:kidney | 0.22 ± 0.15 | 0.27 ± 0.20 | 0.07 ± 0.02 | 0.14 ± 0.04 | 0.26 ± 0.13 |

TABLE 8
Biodistribution data and tumor-to-background contrast ratios of ⁶⁸Ga-labeled HTK03089
and HTK03090 in mice bearing PSMA-expressing LNCAP cancer xenografts.

| | ⁶⁸Ga-HTK03090 | | ⁶⁸Ga-HTK03089 | |
|---|---|---|---|---|
| Tissue | 1 h | 3 h | 1 h | 3 h |
| (% ID/g ) | (n = 5) | (n = 5) | (n = 5) | (n = 4) |
| Blood | 16.8 ± 2.14 | 13.6 ± 1.23 | 6.69 ± 0.27 | 2.52 ± 0.69 |
| Urine | 21.5 ± 9.93 | 53.9 ± 14.6 | 47.9 ± 46.6 | 88.5 ± 45.6 |
| Fat | 1.87 ± 0.80 | 1.30 ± 0.13 | 2.14 ± 0.29 | 1.28 ± 0.25 |
| Seminal | 0.90 ± 0.15 | 0.89 ± 0.05 | 0.66 ± 0.24 | 0.24 ± 0.05 |
| Testes | 1.95 ± 0.20 | 2.08 ± 0.42 | 1.35 ± 0.14 | 0.73 ± 0.16 |
| Intestine | 1.14 ± 0.13 | 0.98 ± 0.16 | 0.76 ± 0.05 | 0.45 ± 0.10 |
| Stomach | 0.59 ± 0.13 | 0.63 ± 0.12 | 0.34 ± 0.07 | 0.19 ± 0.04 |
| Spleen | 2.00 ± 0.34 | 1.93 ± 0.55 | 8.56 ± 3.07 | 2.56 ± 0.89 |
| Liver | 3.37 ± 0.85 | 2.73 ± 0.90 | 1.33 ± 0.27 | 0.73 ± 0.41 |
| Pancreas | 1.36 ± 0.22 | 1.27 ± 0.13 | 1.00 ± 0.07 | 0.47 ± 0.07 |
| Adrenal glands | 3.99 ± 1.94 | 2.89 ± 0.28 | 4.57 ± 1.06 | 2.76 ± 0.44 |
| Kidneys | 24.3 ± 3.03 | 30.0 ± 8.66 | 91.1 ± 7.42 | 96.4 ± 21.9 |
| Lungs | 7.68 ± 1.33 | 6.00 ± 0.88 | 4.68 ± 0.35 | 2.14 ± 0.42 |
| Heart | 3.95 ± 0.72 | 3.00 ± 0.41 | 1.73 ± 0.14 | 0.68 ± 0.16 |
| Tumor | 8.54 ± 0.74 | 16.9 ± 3.16 | 21.7 ± 2.48 | 31.2 ± 2.11 |
| Muscle | 1.23 ± 0.15 | 1.04 ± 0.12 | 0.79 ± 0.06 | 0.29 ± 0.04 |
| Bone | 0.71 ± 0.10 | 0.64 ± 0.03 | 0.41 ± 0.08 | 0.15 ± 0.05 |
| Brain | 0.20 ± 0.02 | 0.16 ± 0.02 | 0.09 ± 0.01 | 0.04 ± 0.02 |
| Tail | 3.77 ± 0.91 | 3.75 ± 1.53 | 2.54 ± 0.33 | 1.01 ± 0.23 |
| Thyroid | 3.36 ± 0.24 | 2.76 ± 0.21 | 2.32 ± 0.10 | 0.98 ± 0.25 |
| Salivary | 2.48 ± 0.36 | 2.15 ± 0.37 | 3.95 ± 0.32 | 1.45 ± 0.33 |
| Lacrimal | 0.16 ± 0.07 | 0.10 ± 0.13 | 0.05 ± 0.03 | 0.00 ± 0.00 |
| Tumor:Blood | 0.52 ± 0.10 | 1.25 ± 0.32 | 3.25 ± 0.43 | 13.1 ± 3.76 |
| Tumor:Muscle | 7.09 ± 1.37 | 16.6 ± 4.11 | 27.4 ± 4.01 | 108 ± 13.6 |

U.S. provisional application No. 62/575,460 filed 22 Oct. 2017 is incorporated herein by reference in its entirety. To the extent that there may be disagreement between definitions provided in this application and those provided in a document incorporated by reference, the definitions in this application shall override those in a document incorporated by reference.

The present invention has been described with regard to one or more embodiments. However, it will be apparent to persons skilled in the art that a number of variations and modifications can be made without departing from the scope of the invention as defined in the claims

TABLE 9
Biodistribution data and tumor-to-background contrast ratios of ⁶⁸Ga-labeled
HTK03024, HTK03055, HTK03056, and HTK03058 in mice bearing
PSMA-expressing LNCAP cancer xenografts.

| | ⁶⁸Ga-HTK03024 | | ⁶⁸Ga-HTK03058 | | ⁶⁸Ga-HTK03055 | | ⁶⁸Ga-HTK03056 | |
|---|---|---|---|---|---|---|---|---|
| Tissue | 1 h | 3 h | 1 h | 3 h | 1 h | 3 h | 1 h | 3 h |
| (% ID/g) | (n = 2) | (n = 4) | (n = 5) | (n = 5) | (n = 5) | (n = 5) | (n = 5) | (n = 5) |
| Blood | 27.0 ± 5.63 | 23.9 ± 1.03 | 26.7 ± 3.02 | 21.2 ± 2.21 | 22.1 ± 2.04 | 17.4 ± 1.15 | 4.68 ± 1.09 | 1.17 ± 0.36 |
| Urine | 1.48 ± 0.71 | 3.68 ± 1.74 | 3.97 ± 2.56 | 6.15 ± 3.78 | 14.6 ± 10.9 | 33.8 ± 22.3 | 203 ± 88.0 | 275 ± 158 |
| Fat | 1.83 ± 0.44 | 3.53 ± 1.48 | 2.13 ± 0.57 | 2.47 ± 1.61 | 2.20 ± 0.41 | 2.12 ± 0.69 | 0.87 ± 0.53 | 0.58 ± 0.13 |
| Seminal | 1.27 ± 0.18 | 1.63 ± 0.13 | 1.31 ± 0.38 | 1.15 ± 0.16 | 1.19 ± 0.18 | 1.07 ± 0.12 | 0.74 ± 0.39 | 0.29 ± 0.08 |
| Testes | 2.98 ± 0.30 | 3.07 ± 0.20 | 2.74 ± 0.38 | 3.02 ± 0.80 | 2.54 ± 0.34 | 2.63 ± 0.49 | 0.98 ± 0.22 | 0.56 ± 0.19 |
| Intestine | 1.86 ± 0.06 | 1.66 ± 0.14 | 1.64 ± 0.27 | 1.63 ± 0.18 | 1.52 ± 0.21 | 1.29 ± 0.18 | 0.60 ± 0.14 | 0.34 ± 0.04 |
| Stomach | 0.64 ± 0.05 | 0.70 ± 0.10 | 0.58 ± 0.20 | 0.79 ± 0.23 | 0.62 ± 0.09 | 0.59 ± 0.13 | 0.27 ± 0.10 | 0.14 ± 0.03 |
| Spleen | 2.72 ± 0.69 | 2.62 ± 0.93 | 3.13 ± 1.05 | 2.11 ± 0.44 | 3.05 ± 0.63 | 3.07 ± 0.78 | 3.92 ± 0.88 | 2.10 ± 1.16 |
| Liver | 3.71 ± 0.38 | 3.86 ± 0.63 | 3.87 ± 1.00 | 2.80 ± 0.46 | 3.35 ± 1.08 | 2.96 ± 0.56 | 1.02 ± 0.34 | 0.43 ± 0.08 |
| Pancreas | 1.86 ± 0.04 | 1.86 ± 0.20 | 2.00 ± 0.17 | 1.96 ± 0.20 | 2.02 ± 0.32 | 1.74 ± 0.30 | 0.85 ± 0.15 | 0.41 ± 0.09 |
| Adrenal | 4.32 ± 0.25 | 6.37 ± 1.54 | 4.20 ± 1.21 | 4.46 ± 1.42 | 5.51 ± 1.56 | 6.45 ± 1.03 | 3.78 ± 0.46 | 3.95 ± 2.28 |
| glands | | | | | | | | |
| Kidneys | 9.11 ± 1.06 | 12.3 ± 0.86 | 14.7 ± 2.13 | 17.5 ± 3.83 | 31.1 ± 4.40 | 35.6 ± 7.23 | 123 ± 30.4 | 31.1 ± 15.2 |
| Lungs | 9.51 ± 0.55 | 11.6 ± 0.82 | 10.2 ± 2.20 | 8.21 ± 1.21 | 8.37 ± 1.10 | 7.50 ± 1.05 | 3.28 ± 0.85 | 1.39 ± 0.53 |
| Heart | 6.53 ± 1.30 | 5.56 ± 0.43 | 6.09 ± 0.97 | 4.85 ± 0.67 | 5.20 ± 0.59 | 3.89 ± 0.40 | 1.29 ± 0.34 | 0.59 ± 0.26 |
| Tumor | 7.34 ± 0.10 | 15.0 ± 2.19 | 11.2 ± 3.33 | 14.2 ± 1.99 | 17.2 ± 1.67 | 30.1 ± 3.12 | 25.9 ± 3.21 | 32.9 ± 10.9 |
| Muscle | 1.10 ± 0.18 | 1.55 ± 0.15 | 1.37 ± 0.17 | 1.33 ± 0.24 | 1.60 ± 0.19 | 1.26 ± 0.18 | 0.53 ± 0.11 | 0.46 ± 0.30 |
| Bone | 1.26 ± 0.08 | 1.66 ± 0.39 | 1.04 ± 0.26 | 1.54 ± 0.36 | 1.59 ± 0.44 | 2.11 ± 0.62 | 0.76 ± 0.14 | 1.93 ± 1.51 |
| Brain | 0.31 ± 0.04 | 0.34 ± 0.02 | 0.36 ± 0.06 | 0.30 ± 0.06 | 0.29 ± 0.04 | 0.28 ± 0.06 | 0.10 ± 0.03 | 0.12 ± 0.05 |
| Tail | 7.58 ± 4.49 | 4.03 ± 0.43 | 8.87 ± 4.26 | 6.86 ± 2.42 | 4.42 ± 0.76 | 3.52 ± 0.65 | 1.43 ± 0.34 | 0.54 ± 0.22 |
| Thyroid | 4.39 ± 0.01 | 4.19 ± 0.48 | 4.95 ± 0.79 | 3.72 ± 0.33 | 4.20 ± 0.53 | 3.40 ± 0.56 | 1.49 ± 0.44 | 0.72 ± 0.29 |
| Salivary | 3.31 ± 0.79 | 4.66 ± 0.58 | 3.90 ± 0.70 | 3.45 ± 0.40 | 3.87 ± 0.70 | 3.57 ± 1.07 | 3.34 ± 0.89 | 2.06 ± 1.16 |
| Lacrimal | 0.93 ± 0.36 | 3.57 ± 1.56 | 0.69 ± 0.26 | 1.44 ± 0.37 | 1.82 ± 1.46 | 4.14 ± 3.82 | 1.35 ± 0.43 | 3.01 ± 1.26 |
| Tumor:Blood | 0.28 ± 0.05 | 0.63 ± 0.11 | 0.42 ± 0.11 | 0.67 ± 0.07 | 0.78 ± 0.04 | 1.75 ± 0.28 | 5.67 ± 0.97 | 30.4 ± 13.2 |
| Tumor:Muscle | 6.74 ± 1.00 | 9.69 ± 1.51 | 8.04 ± 1.69 | 10.8 ± 1.56 | 10.8 ± 1.15 | 24.5 ± 5.08 | 49.7 ± 5.98 | 98.6 ± 61.8 |
| Tumor:kidney | 0.81 ± 0.11 | 1.21 ± 0.10 | 0.75 ± 0.17 | 0.83 ± 0.11 | 0.56 ± 0.06 | 0.88 ± 0.22 | 0.22 ± 0.04 | 1.22 ± 0.53 |

TABLE 10
Biodistribution data and tumor-to-background contrast ratios of ⁶⁸Ga-labeled
HTK03082, HTK03085, HTK03086, and HTK03087 in mice bearing
PSMA-expressing LNCAP cancer xenografts.

| | ⁶⁸Ga-HTK03082 | | ⁶⁸Ga-HTK03086 | | ⁶⁸Ga-HTK03087 | | ⁶⁸Ga-HTK03085 | |
|---|---|---|---|---|---|---|---|---|
| Tissue | 1 h | 3 h | 1 h | 3 h | 1 h | 3 h | 1 h | 3 h |
| (% ID/g) | (n = 5) | (n = 5) | (n = 5) | (n = 5) | (n = 5) | (n = 5) | (n = 5) | (n = 4) |
| Blood | 1.37 ± 0.23 | 0.25 ± 0.04 | 12.1 ± 0.60 | 6.60 ± 0.84 | 1.74 ± 0.11 | 0.52 ± 0.08 | 5.73 ± 0.93 | 2.10 ± 0.48 |
| Urine | 349 ± 209 | 191 ± 83.1 | 82.9 ± 62.5 | 1.30 ± 47.9 | 196 ± 50.9 | 339 ± 355 | 181 ± 97.2 | 141 ± 54.3 |
| Fat | 0.61 ± 0.09 | 0.21 ± 0.01 | 1.37 ± 0.17 | 0.95 ± 0.12 | 0.77 ± 0.14 | 0.40 ± 0.11 | 1.36 ± 0.28 | 0.70 ± 0.13 |
| Seminal | 0.81 ± 0.94 | 0.12 ± 0.08 | 0.71 ± 0.10 | 0.47 ± 0.09 | 2.40 ± 2.83 | 1.44 ± 3.04 | 1.42 ± 2.17 | 0.21 ± 0.08 |
| Testes | 0.42 ± 0.05 | 0.13 ± 0.08 | 1.54 ± 0.18 | 1.30 ± 0.30 | 0.48 ± 0.06 | 0.17 ± 0.05 | 1.03 ± 0.12 | 0.60 ± 0.09 |
| Intestine | 0.33 ± 0.07 | 0.19 ± 0.02 | 0.91 ± 0.07 | 0.71 ± 0.07 | 0.32 ± 0.06 | 0.23 ± 0.10 | 0.61 ± 0.10 | 0.39 ± 0.08 |
| Stomach | 0.10 ± 0.03 | 0.05 ± 0.01 | 0.34 ± 0.04 | 0.27 ± 0.08 | 0.14 ± 0.05 | 0.07 ± 0.04 | 0.24 ± 0.05 | 0.16 ± 0.09 |
| Spleen | 1.12 ± 0.19 | 0.24 ± 0.08 | 2.18 ± 0.51 | 1.58 ± 0.37 | 2.42 ± 0.79 | 0.80 ± 0.31 | 3.90 ± 1.57 | 1.05 ± 0.09 |
| Liver | 0.39 ± 0.09 | 0.17 ± 0.02 | 1.77 ± 0.27 | 1.40 ± 0.35 | 0.44 ± 0.06 | 0.31 ± 0.19 | 1.01 ± 0.29 | 0.61 ± 0.08 |
| Pancreas | 0.47 ± 0.26 | 0.09 ± 0.03 | 1.16 ± 0.11 | 0.70 ± 0.11 | 0.45 ± 0.02 | 0.14 ± 0.03 | 0.86 ± 0.09 | 0.35 ± 0.09 |
| Adrenal | 2.19 ± 0.76 | 0.38 ± 0.06 | 3.28 ± 0.54 | 1.80 ± 0.39 | 2.95 ± 0.92 | 0.90 ± 0.36 | 3.57 ± 1.09 | 1.37 ± 0.48 |
| glands | | | | | | | | |
| Kidneys | 81.6 ± 12.6 | 22.3 ± 9.97 | 54.7 ± 4.84 | 48.5 ± 10.7 | 102 ± 8.91 | 56.1 ± 20.3 | 85.8 ± 8.36 | 63.4 ± 16.6 |
| Lungs | 1.39 ± 0.27 | 0.28 ± 0.07 | 5.43 ± 0.65 | 3.49 ± 0.64 | 1.75 ± 0.09 | 0.53 ± 0.12 | 3.67 ± 0.72 | 1.50 ± 0.26 |
| Heart | 0.40 ± 0.07 | 0.07 ± 0.02 | 2.76 ± 0.25 | 1.53 ± 0.21 | 0.52 ± 0.07 | 0.16 ± 0.03 | 1.46 ± 0.27 | 0.52 ± 0.09 |
| Tumor | 13.6 ± 2.16 | 14.5 ± 2.12 | 17.8 ± 2.89 | 28.2 ± 5.44 | 20.7 ± 3.79 | 20.4 ± 5.92 | 18.8 ± 3.35 | 28.9 ± 4.27 |
| Muscle | 0.33 ± 0.15 | 0.11 ± 0.11 | 0.94 ± 0.02 | 0.57 ± 0.09 | 0.31 ± 0.05 | 0.08 ± 0.02 | 0.58 ± 0.08 | 0.31 ± 0.08 |
| Bone | 0.15 ± 0.06 | 0.01 ± 0.01 | 0.72 ± 0.19 | 0.33 ± 0.16 | 0.16 ± 0.02 | 0.03 ± 0.03 | 0.34 ± 0.08 | 0.13 ± 0.04 |
| Brain | 0.03 ± 0.01 | 0.01 ± 0.00 | 0.15 ± 0.01 | 0.09 ± 0.02 | 0.04 ± 0.01 | 0.01 ± 0.00 | 0.08 ± 0.01 | 0.04 ± 0.01 |
| Tail | 0.84 ± 0.14 | 0.24 ± 0.14 | 3.39 ± 0.59 | 1.50 ± 0.17 | 0.91 ± 0.12 | 0.27 ± 0.05 | 1.79 ± 0.36 | 0.60 ± 0.15 |
| Thyroid | 0.58 ± 0.11 | 0.16 ± 0.09 | 2.24 ± 0.15 | 1.39 ± 0.16 | 0.74 ± 0.04 | 0.24 ± 0.05 | 1.70 ± 0.25 | 0.64 ± 0.10 |
| Salivary | 1.17 ± 0.27 | 0.20 ± 0.07 | 2.51 ± 0.18 | 1.39 ± 0.13 | 1.76 ± 0.16 | 0.47 ± 0.07 | 2.61 ± 0.59 | 1.03 ± 0.33 |
| Lacrimal | 0.08 ± 0.03 | 0.01 ± 0.01 | 0.24 ± 0.05 | 0.07 ± 0.04 | 0.06 ± 0.05 | 0.00 ± 0.00 | 0.11 ± 0.06 | 0.04 ± 0.07 |
| Tumor:Blood | 10.1 ± 1.91 | 58.1 ± 12.6 | 1.47 ± 0.28 | 4.33 ± 1.06 | 11.9 ± 1.92 | 39.2 ± 8.42 | 3.33 ± 0.75 | 14.2 ± 2.77 |
| Tumor:Muscle | 49.2 ± 24.2 | 223 ± 122 | 18.8 ± 2.968 | 50.5 ± 13.5 | 66.6 ± 9.63 | 254 ± 88.9 | 33.2 ± 7.91 | 95.8 ± 20.9 |
| Tumorkidney | 0.17 ± 0.02 | 1.12 ± 1.30 | 0.32 ± 0.04 | 0.59 ± 0.10 | 0.20 ± 0.02 | 0.40 ± 0.16 | 0.22 ± 0.03 | 0.48 ± 0.11 |

1. Carter, R. E.; Feldman, A. R.; Coyle, J. T. Prostate-specific membrane antigen is a hydrolase with substrate and pharmacologic characteristics of a neuropeptidase. Proc. Natl. Acad. Sci. U.S.A 1996, 93, 749-753.
2. Silver, D. A.; Pellicer, I.; Fair, W. R.; Heston, W. D.; Cordon-Cardo, C. Prostate-specific membrane antigen expression in normal and malignant human tissues. Clin. Cancer Res. 1997, 3, 81-85.
3. Sokoloff, R. L.; Norton, K. C.; Gasior, C. L.; Marker, K. M.; Grauer, L. S. A dual-monoclonal sandwich assay for prostate-specific membrane antigen: levels in tissues, seminal fluid and urine. Prostate 2000, 43, 150-157.
4. Bander, N. H.; Milowsky, M. I.; Nanus, D. M.; Kostakoglu, L.; Vallabhajosula, S.; Goldsmith, S. J. Phase I trial of ¹⁷⁷lutetium-labeled J591, a monoclonal antibody to prostate-specific membrane antigen, in patients with androgen-independent prostate cancer. J. Clin. Oncol. 2005, 23, 4591-4601.
5. Afshar-Oromieh, A.; Haberkorn, U.; Zechmann, C.; Armor, T.; Mier, W.; Spohn, F.; Debus, N.; Holland-Letz, T.; Babich, J.; Kratochwil, C. Repeated PSMA-targeting radioligand therapy of metastatic prostate cancer with ¹³¹I-MIP-1095. Eur. J. Nucl. Med. Mol. Imaging 2017, 44, 950-959.
6. Heck, M. M.; Retz, M.; D'Alessandria, C.; Rauscher, I.; Scheidhauer, K.; Maurer, T.; Storz, E.; Janssen, F.; Schottelius, M.; Wester, H. J.; Gschwend, J. E.; Schwaiger, M.; Tauber, R.; Eiber, M. Systemic radioligand therapy with ¹⁷⁷Lu labeled prostate specific membrane antigen ligand for imaging and therapy in patients with metastatic castration resistant prostate cancer. J. Urol. 2016, 196, 382-391.
7. Kratochwil, C.; Giesel, F. L.; Stefanova, M.; Benesova, M.; Bronzel, M.; Afshar-Oromieh, A.; Mier, W.; Eder, M.; Kopka, K.; Haberkorn, U. PSMA-targeted radionuclide therapy of metastatic castration-resistant prostate cancer with ¹⁷⁷Lu-labeled PSMA-617. J. Nucl. Med. 2016, 57, 1170-1176.
8. Rahbar, K.; Bode, A.; Weckesser, M.; Avramovic, N.; Claesener, M.; Stegger, L.; Bogemann, M. Radioligand therapy with ¹⁷⁷Lu-PSMA-617 as a novel therapeutic option in patients with metastatic castration resistant prostate cancer. Clin. Nucl. Med. 2016, 41, 522-528.
9. Fendler, W. P.; Reinhardt, S.; Ilhan, H.; Delker, A.; Boning, G.; Gildehaus, F. J.; Stief, C.; Bartenstein, P.; Gratzke, C.; Lehner, S.; Rominger, A. Preliminary experience with dosimetry, response and patient reported outcome after ¹⁷⁷Lu-PSMA-617 therapy for metastatic castration-resistant prostate cancer. Oncotarget 2017, 8, 3581-3590.
10. Rahbar, K.; Ahmadzadehfar, H.; Kratochwil, C.; Haberkorn, U.; Schafers, M.; Essler, M.; Baum, R. P.; Kulkarni, H. R.; Schmidt, M.; Drzezga, A.; Bartenstein, P.; Pfestroff, A.; Luster, M.; Lutzen, U.; Marx, M.; Prasad, V.; Brenner, W.; Heinzel, A.; Mottaghy, F. M.; Ruf, J.; Meyer, P. T.; Heuschkel, M.; Eveslage, M.; Bogemann, M.; Fendler, W. P.; Krause, B. J. German multicenter study investigating ¹⁷⁷Lu-PSMA-617 radioligand therapy in advanced prostate cancer patients. J. Nucl. Med. 2017, 58, 85-90.
11. Ahmadzadehfar, H.; Wegen, S.; Yordanova, A.; Fimmers, R.; Kurpig, S.; Eppard, E.; Wei, X.; Schlenkhoff, C.; Hauser, S.; Essler, M. Overall survival and response pattern of castration-resistant metastatic prostate cancer to multiple cycles of radioligand therapy using [¹⁷⁷Lu]Lu-PSMA-617. Eur. J. Nucl. Med. Mol. Imaging 2017, 44, 1448-1454.
12. Brauer, A.; Grubert, L. S.; Roll, W.; Schrader, A. J.; Schafers, M.; Bogemann, M.; Rahbar, K. ¹⁷⁷Lu-PSMA-617 radioligand therapy and outcome in patients with metastasized castration-resistant prostate cancer. Eur. J. Nucl. Med. Mol. Imaging 2017, 44, 1663-1670.
13. Yadav, M. P.; Ballal, S.; Tripathi, M.; Damle, N. A.; Sahoo, R. K.; Seth, A.; Bal, C. ¹⁷⁷Lu-DKFZ-PSMA-617 therapy in metastatic castration resistant prostate cancer: safety, efficacy, and quality of life assessment. Eur. J. Nucl. Med. Mol. Imaging 2017, 44, 81-91.
14. Hofman, M. S.; Violet, J.; Hicks, R. J.; Ferdinandus, J.; Thang, S. P.; Akhurst, T.; Iravani, A.; Kong, G.; Kumar, A. R.; Murphy, D. G.; Eu, P.; Jackson, P.; Scalzo, M.; Williams, S. G.; Sandhu, S. [¹⁷⁷Lu]-PSMA-617 radionuclide treatment in patients with metastatic castration-resistant prostate cancer (LuPSMA trial): a single-centre, single-arm, phase 2 study. Lancet Oncol. 2018, 19, 825-833.
15. Kratochwil, C.; Bruchertseifer, F.; Giesel, F. L.; Weis, M.; Verburg, F. A.; Mottaghy, F.; Kopka, K.; Apostolidis, C.; Haberkorn, U.; Morgenstern, A. ²²⁵Ac-PSMA-617 for PSMA-targeted α-radiation therapy of metastatic castration-resistant prostate cancer. J. Nucl. Med. 2016, 57, 1941-1944.
16. Benesova, M.; Schafer, M.; Bauder-Wust, U.; Afshar-Oromieh, A.; Kratochwil, C.; Mier, W.; Haberkorn, U.; Kopka, K.; Eder, M. Preclinical evaluation of a tailor-made DOTA-conjugated PSMA inhibitor with optimized linker moiety for imaging and endoradiotherapy of prostate cancer. J. Nucl. Med. 2015, 56, 914-920.
17. Benesova, M.; Bauder-Wust, U.; Schafer, M.; Klika, K. D.; Mier, W.; Haberkorn, U.; Kopka, K.; Eder, M. Linker modification strategies to control the prostate-specific membrane antigen (PSMA)-targeting and pharmacokinetic properties of DOTA-conjugated PSMA inhibitors. J. Med. Chem. 2016, 59, 1761-1775.
18. Kuo, H. T.; Pan, J.; Zhang, Z.; Lau, J.; Merkens, H.; Zhang, C.; Colpo, N.; Lin, K. S.; Benard, F. Effects of linker modification on tumor-to-kidney contrast of ⁶⁸Ga-labeled PSMA-targeted imaging probes. Mol. Pharmaceutics 2018, doi: 10.1021/acs.molpharmaceut.8b00499
19. Meckel, M.; Kubicek, V.; Hermann, P.; Miederer, M.; Rosch, F. A DOTA based bisphosphonate with an albumin binding moiety for delayed body clearance for bone targeting. Nucl. Med. Biol. 2016, 43, 670-678.
20. Harada, N.; Kimura, H.; Onoe, S.; Watanabe, H.; Matsuoka, D.; Arimitsu, K.; Ono, M.; Saji, H. Synthesis and biological evaluation of novel ¹⁸F-labeled probes targeting prostate-specific membrane antigen for positron emission tomography of prostate cancer. J. Nucl. Med. 2016, 57, 1978-1984.
21. Chatalic, K. L. S.; Heskamp, S.; Konijnenberg, M.; Molkenboer-Kuenen, J. D. M.; Franssen, G. M.; Clahsen-van Groningen, M. C.; Schottelius, M.; Wester, H. J.; van Weerden, W. M.; Boerman, O. C.; de Jong, M. Towards personalized treatment of prostate cancer: PSMA I&T, a promising prostate-specific membrane antigen-targeted theranostic agent. Theranostics 2016, 6, 849-861.
22. Dumelin, C. E.; Trussel, S.; Buller, F.; Trachsel, E.; Bootz, F.; Zhang, Y.; Mannocci, L.; Beck, S. C.; Drumea-Mirancea, M.; Seeliger, M. W.; Baltes, C.; Muggler, T.; Kranz, F.; Rudin, M.; Melkko, S.; Scheuermann, J.; Neri, D. A portable albumin binder from a DNA-encoded chemical library. Angew. Chem. Int. Ed. 2008, 47, 3196-3201.
23. Muller, C.; Struthers, H.; Winiger, C.; Zhernosekov, K.; Schibli, R. DOTA conjugate with an albumin-binding entity enables the first folic acid-targeted ¹⁷⁷Lu-radionuclide tumor therapy in mice. J. Nucl. Med. 2013, 54, 124-131.
24. Choy, C. J.; Ling, X.; Geruntho, J. J.; Beyer, S. K.; Latoche, J. D.; Langton-Webster, B.; Anderson, C. J.; Berkman, C. E. ¹⁷⁷Lu-Labeled phosphoramidate-based PSMA inhibitors: The effect of an albumin binder on biodistribution and therapeutic efficacy in prostate tumor-bearing mice. Theranostics 2017, 7, 1928-1939.
25. Kelly, J. M.; Amor-Coarasa, A.; Nikolopoulou, A.; Wustemann, T.; Barelli, P.; Kim, D.; Williams, C.; Zheng, X.; Bi, C.; Hu, B.; Warren, J. D.; Hage, D. S.; DiMagno, S. G.; Babich, J. W. Dual-target binding ligands with modulated pharmacokinetics for endoradiotherapy of prostate cancer. J. Nucl. Med. 2017, 58, 1442-1449.
26. Benesova, M.; Umbricht, C. A.; Schibli, R.; Muller, C. Albumin-binding PSMA ligands: optimization of the tissue distribution profile. Mol. Pharmaceutics 2018, 15, 934-946.
27. Umbricht, C. A.; Benesova, M.; Schibli, R.; Muller, C. Preclinical development of novel PSMA-targeting radioligands: Modulation of albumin-binding properties to improve prostate cancer therapy. Mol. Pharmaceutics 2018, 15, 2297-2306.
28. Kelly, J.; Amor-Coarasa, A.; Ponnala, S.; Nikolopoulou, A.; Williams, C.; Schlyer, D.; Zhao, Y.; Kim, D.; Babich, J. W. Trifunctional PSMA-targeting constructs for prostate cancer with unprecedented localization to LNCaP tumors. Eur. J. Nucl. Med. Mol. Imaging 2018, doi: 10.1007/s00259-018-4004-5.
29. Liu, Z.; Chen, X. Simple Bioconjugate chemistry serves great clinical advances: albumin as a versatile platform for diagnosis and precision therapy. Chem. Soc. Rev. 2016, 45, 1432-1456.
30. Apostolidis, C.; Molinet, R.; Rasmussen, G.; Morgenstern, A. Production of Ac-225 from Th-229 for targeted α therapy. Anal. Chem. 2005, 77, 6288-6291.
31. Walsh, K. M. Brookhaven National Laboratory: Radioisotopes for medical imaging and disease treatment. J. Nucl. Med. 2017, 58, 11N-12N.
37. Stabin M G, Sparks R B, Crowe E. OLINDA/EXM: the second-generation personal computer software for internal dose assessment in nuclear medicine. J Nucl Med. 2005; 46:1023-1027.
38. Keenan M A, Stabin M G, Segars W P, Fernald M J. RADAR realistic animal model series for dose assessment. J Nucl Med. 2010; 51:471-476.
39. Stabin M G, Xu X G, Emmons M A, Segars W P, Shi C, Fernald M J. RADAR reference adult, pediatric, and pregnant female phantom series for internal and external dosimetry. J Nucl Med. 2012; 53:1807-1813.
40. Stabin M G, Konijnenberg M W. Re-evaluation of absorbed fractions for photons and electrons in spheres of various sizes. J Nucl Med. 2000; 41:149-160.
41. Kirschner A S, Ice R D, Beierwaltes W H. Radiation dosimetry of ¹³¹I-19-iodocholesterol: the pitfalls of using tissue concentration data. The author's reply. J Nucl Med. 1975; 16:248-249.
42. Wessels B W, Bolch W E, Bouchet L G, et al. Bone marrow dosimetry using blood-based models for radiolabeled antibody therapy: a multiinstitutional comparison. J Nucl Med. 2004; 45(10):1725-1733.

What is claimed is:

1. A compound which is of Formula I-a or Formula I-b, or is a salt or solvate of Formula I-a or Formula I-b: 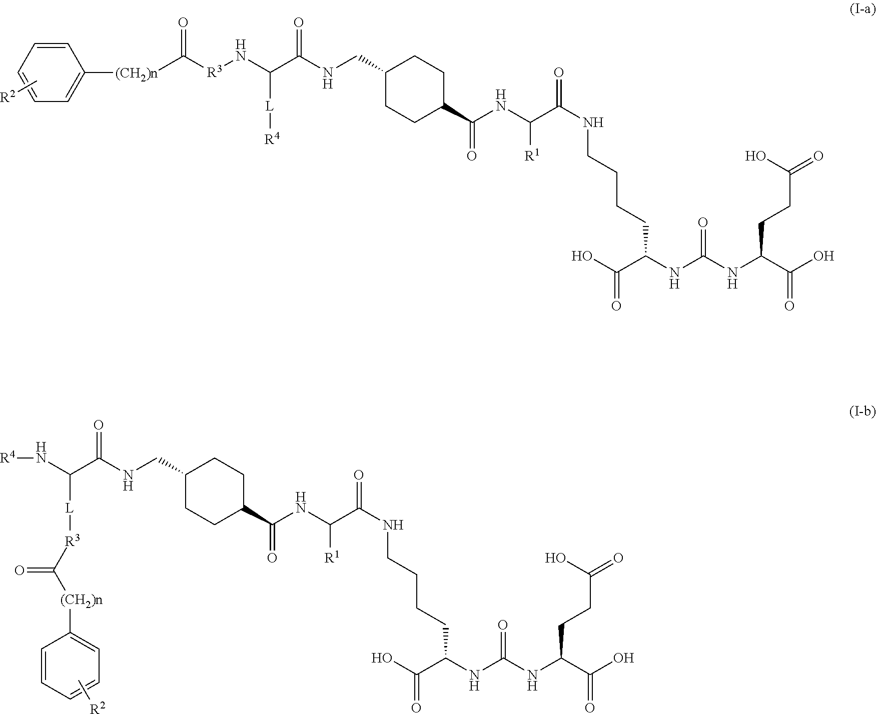
wherein:
R¹ is 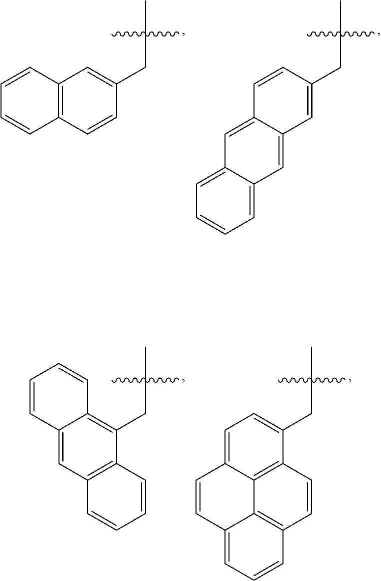 [SMILES: *Cc1ccc2ccccc2c1 ; *Cc1ccc2cc3ccccc3cc2c1 ; *Cc1c2ccccc2cc2ccccc12 ; *Cc1ccc2ccc3cccc4ccc1c2c34]
or —(CH₂)₅CH₃;
R² is I, Br, F, Cl, H, OH, OCH₃, NH₂, NO₂ or CH₃;
R³ is 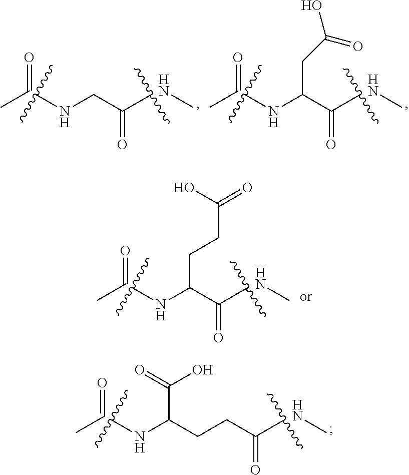
L is —CH₂NH—, —(CH₂)₂NH—, —(CH₂)₃NH—, or —(CH₂)₄NH—;
R⁴ is a radiometal chelator optionally bound by radiometal X; and
n is 1-3.

2. The compound of claim 1, which is of Formula I-a or is a salt or solvate of Formula I-a.

3. The compound of claim 1, which is of Formula I-b or is a salt or solvate of Formula I-b.

4. The compound of any one of claims 1 to 3, wherein R¹ is

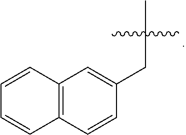 [SMILES: *Cc1ccc2ccccc2c1]

5. The compound of any one of claims 1 to 3, wherein R¹ is

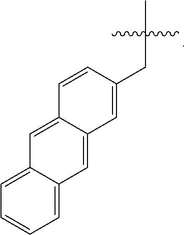 [SMILES: *Cc1ccc2cc3ccccc3cc2c1]

6. The compound of any one of claims 1 to 3, wherein R¹ is

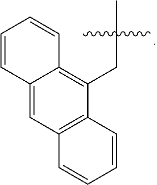 [SMILES: *Cc1c2ccccc2cc2ccccc12]

7. The compound of any one of claims 1 to 3, wherein R¹ is

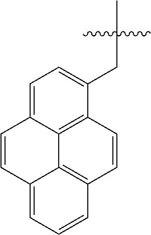 [SMILES: *Cc1ccc2ccc3cccc4ccc1c2c34]

8. The compound of any one of claims 1 to 3, wherein R¹ is —(CH₂)₅CH₃.

9. The compound of any one of claims 1 to 8, wherein R¹ forms the side chain of an L-amino acid residue.

10. The compound of any one of claims 1 to 8, wherein R¹ forms the side chain of a D-amino acid residue.

11. The compound of any one of claims 1 to 3, wherein R¹ is

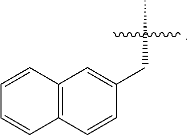

12. The compound of any one of claims 1 to 11, wherein R² is in para position.

13. The compound of any one of claims 1 to 11, wherein R² is in meta position.

14. The compound of any one of claims 1 to 11, wherein R² is in ortho position.

15. The compound of any one of claims 12 to 14, wherein R² is I.

16. The compound of any one of claims 12 to 14, wherein R² is Br.

17. The compound of any one of claims 12 to 14, wherein R² is Cl.

18. The compound of any one of claims 12 to 14, wherein R² is H.

19. The compound of any one of claims 12 to 14, wherein R² is F.

20. The compound of any one of claims 12 to 14, wherein R² is OCH₃.

21. The compound of any one of claims 12 to 14, wherein R² is OH.

22. The compound of any one of claims 12 to 14, wherein R² is NH₂.

23. The compound of any one of claims 12 to 14, wherein R² is NO₂.

24. The compound of any one of claims 12 to 14, wherein R² is CH₃.

25. The compound of any one of claims 1 to 24, wherein R³ is a Gly residue.

26. The compound of any one of claims 1 to 24, wherein R³ is an Asp residue.

27. The compound of any one of claims 1 to 24, wherein R³ is a Glu residue.

28. The compound of any one of claims 1 to 24, wherein R³ is

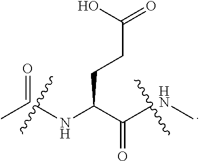

29. The compound of any one of claims 1 to 24, wherein R³ is

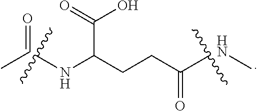

30. The compound of any one of claims 1 to 29, wherein R⁴ is:
DOTA (1,4,7,10-tetraazacyclododecane-1,4,7,10-tetraacetic acid) or a derivative thereof;
TETA (1,4,8,11-tetraazacyclotetradecane-1,4,8,11-tetraacetic acid) or a derivative thereof;
SarAr (1-N-(4-Aminobenzyl)-3,6,10,13,16,19-hexaazabicyclo[6.6.6]-eicosane-1,8-diamine or a derivative thereof;
NOTA (1,4,7-triazacyclononane-1,4,7-triacetic acid) or a derivative thereof;
TRAP (1,4,7-triazacyclononane-1,4,7-tris[methyl(2-carboxyethyl)phosphinic acid) or a derivative thereof;
HBED (N,N0-bis(2-hydroxybenzyl)-ethylenediamine-N,N0-diacetic acid) or a derivative thereof;
2,3-HOPO (3-hydroxypyridin-2-one) or a derivative thereof;
PCTA (3,6,9,15-tetraazabicyclo[9.3.1]-pentadeca-1(15),11,13-triene-3,6,9-triacetic acid) or a derivative thereof;
DFO (desferrioxamine) or a derivative thereof;
DTPA (diethylenetriaminepentaacetic acid) or a derivative thereof;
OCTAPA (N,N0-bis(6-carboxy-2-pyridylmethyl)-ethylenediamine-N,N0-diacetic acid) or a derivative thereof; or
H2-MACROPA (N,N′-bis[(6-carboxy-2-pyridil)methyl]-4,13-diaza-18-crown-6) or a derivative thereof.

31. The compound of claim 30, wherein R⁴ is DOTA.

32. The compound of any one of claims 1 to 31, wherein L is —CH₂NH—.

33. The compound of any one of claims 1 to 31, wherein L is —(CH₂)₂NH—.

34. The compound of any one of claims 1 to 31, wherein L is —(CH₂)₃NH—.

35. The compound of any one of claims 1 to 31, wherein L is —(CH₂)₄NH—.

36. The compound of any one of claims 32 to 35, wherein L forms the side chain of an L-amino acid residue.

37. The compound of any one of claims 32 to 35, wherein L forms the side chain of a D-amino acid residue.

38. The compound of any one of claims 1 to 37, wherein n is 1.

39. The compound of any one of claims 1 to 37, wherein n is 2.

40. The compound of any one of claims 1 to 37, wherein n is 3.

41. The compound of any one of claims 1 to 40, wherein X is absent.

42. The compound of any one of claims 1 to 40, wherein X is ⁶⁴Cu, ⁶⁷Cu, ⁹⁰Y, ¹¹¹In, ^{114m}In, ^{117m}Sn, ¹⁵³Sm, ¹⁴⁹Tb, ¹⁶¹Tb, ¹⁷⁷Lu, ²²⁵Ac, ²¹³Bi, ²²⁴Ra, ²¹²Bi, ²¹²Pb, ²²⁵Ac, ²²⁷Th, ²²³Ra, ⁴⁷Sc, ¹⁸⁶Re or ¹⁸⁸Re.

43. The compound of claim 42, wherein X is ¹⁷⁷Lu.

44. The compound of any one of claims 1 to 40, wherein X is ⁶⁴Cu, ¹¹¹In, ⁸⁹Zr, ⁴⁴Sc, ⁶⁸Ga, ^{99m}Tc, ⁸⁶Y, ¹⁵²Tb or ¹⁵⁵Tb.

45. The compound of claim 44, wherein X is ⁶⁸Ga.

46. A compound which has Formula II or is a salt or solvate of Formula II: 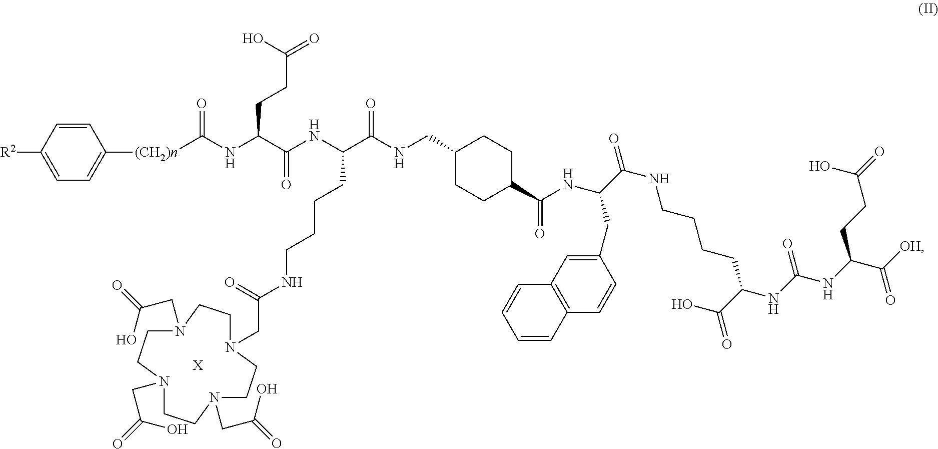
wherein:
R² is I, Br or methyl;
n is 1-3; and
X is absent, ²²⁵Ac or ¹⁷⁷Lu.

47. The compound of claim 46, wherein R² is 1.

48. The compound of claim 46 or 47, wherein n is 3.

49. The compound of any one of claims 46 to 48, wherein X is ¹⁷⁷Lu.

50. A method of imaging prostate specific membrane antigen (PSMA)-expressing cancer in a subject, the method comprising:
administering to the subject a composition comprising the compound of claim 44 or 45 and a pharmaceutically acceptable excipient; and
imaging tissue of the subject.

51. A method of treating prostate specific membrane antigen (PSMA)-expressing cancer in a subject, the method comprising: administering to the subject a composition comprising the compound of any one of claims 42, 43 and 46 to 49 and a pharmaceutically acceptable excipient.

52. The method of claim 50 or 51, wherein the cancer is prostate cancer, renal cancer, breast cancer, thyroid cancer, gastric cancer, colorectal cancer, bladder cancer, pancreatic cancer, lung cancer, liver cancer, brain tumor, melanoma, neuroendocrine tumor, ovarian cancer or sarcoma.
